(19) United States
(12) Patent Application Publication
(10) Pub. No.: US 2025/0353844 A1
(43) Pub. Date: Nov. 20, 2025

(54) TETRAHYDROPYRIDO 3,4-D PYRIMIDINE DERIVATIVES AS KRAS INHIBITORS

(71) Applicant: Bristol-Myers Squibb Company, Princeton, NJ (US)

(72) Inventors: Brian Edward FINK, Yardley, PA (US); Matthew Leo CONDAKES, Brookline, MA (US); Rita Lee CIVIELLO, Killingworth, CT (US); Joanne Jewett BRONSON, Wenham, MA (US)

(21) Appl. No.: 18/873,229

(22) PCT Filed: Jun. 8, 2023
(86) PCT No.: PCT/US2023/068133

§ 371 Date: Dec. 9, 2024

Related U.S. Application Data
(60) Provisional application No. 63/351,134, filed on Jun. 10, 2022.

(51) Int. Cl. C07D 471/04; A61K 31/519; C07D 519/00

(52) U.S. Cl. CPC C07D 471/04; A61K 31/519; C07D 519/00

(57) ABSTRACT

The present disclosure provides KRAS inhibitors. Methods of treating cancers using the compounds are also provided.

### CROSS-REFERENCE TO RELATED APPLICATIONS

This application claims the priority benefit of U.S. Provisional Application No. 63/351,134, filed Jun. 10, 2022 which is incorporated by reference herein in its entirety.

### FIELD

The present disclosure provides KRAS inhibitors. Methods of treating cancers using the inhibitors are also provided.

### BACKGROUND

The KRAS oncogene is a member of the RAS family of GTPases that are involved in numerous cellular signaling processes. KRAS mutations are gain-of-function mutations that are present in up to 30% of all tumors, including as many as 90% of pancreatic cancers. KRAS serves as a molecular switch cycling between inactive (GDP-bound or G12C^{(OFF)}) and active (GTP-bound or G12C^{(ON)}) states to transduce upstream cellular signals received from multiple tyrosine kinases to downstream effectors to regulate a wide variety of processes, including cellular proliferation. Single nucleotide substitutions that result in missense mutations at codons 12 and 13 of the KRAS primary amino acid sequence comprise approximately 40% of KRAS driver mutations in lung adenocarcinoma, with a G12C transversion being the most common activating mutation. KRAS G12C mutations occur in about 13% of lung adenocarcinomas and about 3% of colorectal adenocarcinomas and are also present in cancers of the breast, bladder, cervix, ovaries, pancreas and uterus.

Despite several unsuccessful efforts to target KRAS, compounds that inhibit KRAS activity, including those that disrupt effectors such as guanine nucleotide exchange factors and target KRAS G12C, are highly desirable. Clearly there remains a continued interest and effort to develop inhibitors of KRAS, particularly inhibitors of activating KRAS mutants, such as KRAS G12C.

### SUMMARY

The present disclosure is based, in part, on the discovery that unlike other KRAS G12C inhibitors, compounds of the disclosure target the active, KRAS G12C^{(ON)} form of KRAS G12C protein. By inhibiting the G12C^{ON} form of KRAS, G12C, it is expected that the claimed compounds will decrease a cancer's resistance to KRAS G12C inhibition and/or demonstrate increased potency in the clinic. Without being bound by a theory, the inhibition of G12C^{ON} form of KRAS G12C may be a result of the substituent at position 4 of the tetrahydropyridopyrimidine ring in formula (I).

In a first aspect, the present disclosure provides a compound of formula (I):

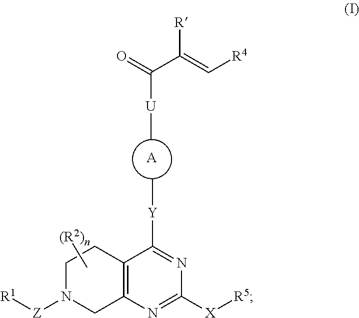

or a pharmaceutically acceptable salt thereof, wherein:
U is a bond or NH;
n is 0, 1, or 2;
Z is a bond, C(O), or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl;
each R² is independently selected from C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, hydroxy, and oxo;
Y is a bond, O, NR^{g}(CR^{e}R^{f})ₘ, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, halo, haloC₁-C₃alkyl, amino, aminoC₁-C₃alkyl, hydroxy, hydroxyC₁-C₃alkyl, and oxo;
R′ is halo;
R⁴ is a five- or six-membered aromatic ring optionally containing one, two, or three heteroatoms independently selected from nitrogen, oxygen, and sulfur; wherein the ring is optionally substituted with one, two, or three substituents independently selected from C₂-C₄alkenyl, C₁-C₃alkyl, cyano, cyanoC₁-C₃alkyl, halo, haloC₁-C₃alkoxy, haloC₁-C₃alkyl, nitro, and oxo;
X is O or NR¹⁶, wherein R¹⁶ is hydrogen or C₁-C₃alkyl;
R⁵ is selected from hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from C₁-C₃alkoxy, C₁-C₃alkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, aminoC₁-C₃alkyl, hydroxy, and hydroxyC₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

In some aspects, Y is a bond.

In some aspects, A is a four- to nine-membered monocyclic or bicyclic bridged or fused saturated ring system optionally containing one or two nitrogen atoms.

In some aspects, A-U is

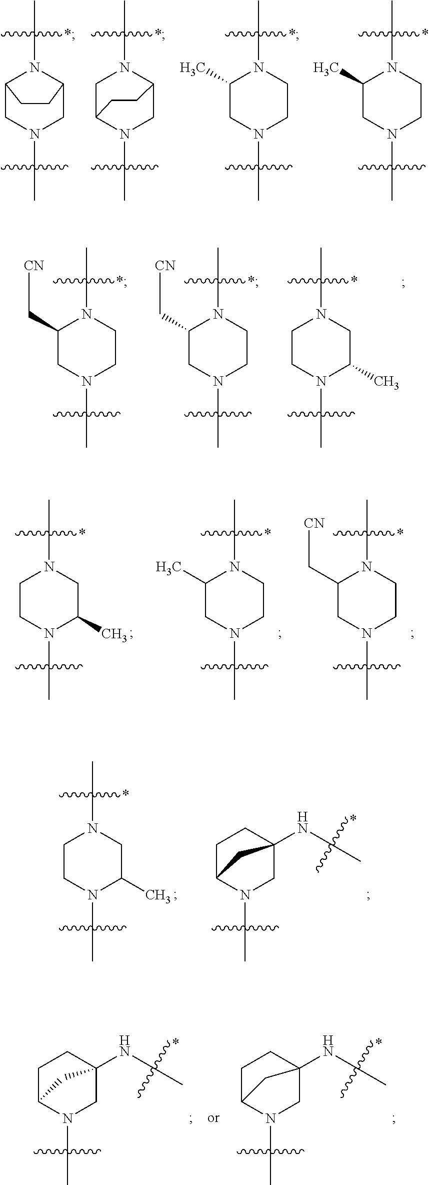

wherein
[symbol] resents the point of attachment to the carbonyl group; and
[symbol] represents the point of attachment to Y.

In some aspects, A-U is

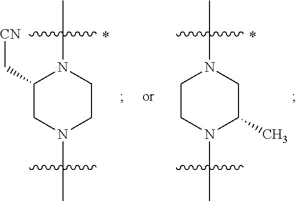

wherein
[symbol] represents the point of attachment to the carbonyl group; and
[symbol] represents the point of attachment to Y.

In some aspects, n is 0.

In some aspects, R⁴ is selected from imidazolyl, isothiazolyl, isoxazolyl, oxazolyl, phenyl, pyridazinyl, pyridinyl, pyrimidinyl, pyrazolyl, thiazolyl, and triazolyl, wherein each ring is optionally substituted with one, two, or three groups independently selected from C₂-C₄alkenyl, C₁-C₃alkyl, halo, haloC₁-C₃alkoxy, haloC₁-C₃alkyl, nitro, and oxo.

In some aspects, R⁴ is selected from imidazolyl, isothiazolyl, isoxazolyl, oxazolyl, pyridinyl, pyrimidinyl, thiazolyl, and triazolyl, wherein each ring is optionally substituted with a methyl or halo.

In some aspects, X is O.

In some aspects, R⁵ is selected from:

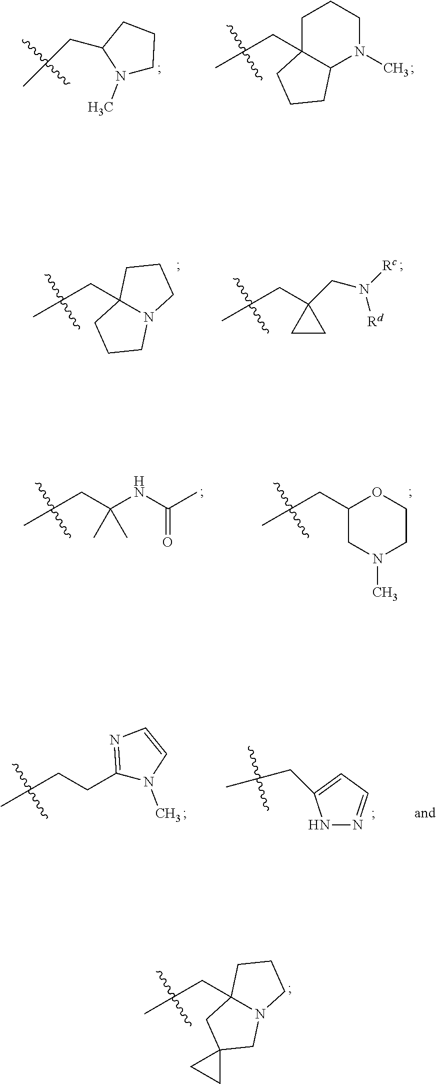

wherein each ring is optionally substituted with 1, 2, or 3 groups independently selected from C₁-C₃alkoxy, C₁-C₃alkoxyC₁-C₃alkyl, C₁-C₃alkyl, benzyl, halo, haloC₁-C₃alkyl, hydroxy, hydroxyC₁-C₃alkyl, and oxo.

In some aspects, R⁵ is —(C₁-C₃alkyl)-R⁶, wherein R⁶ is a three- to five-membered monocyclic ring system, an eight- or nine-membered bicyclic fused saturated ring system, or a ten-membered tricyclic saturated ring system, wherein each ring system optionally contains one nitrogen atom, and wherein each ring system is optionally substituted with one or two groups independently selected from C₁-C₃alkyl, halo, and (4- to 6-membered heterocyclyl)C₁-C₃alkyl; wherein the heterocyclyl part of the (4- to 6-membered heterocyclyl)C₁-C₃alkyl is further optionally substituted with a halo group.

In some aspects, R⁵ is

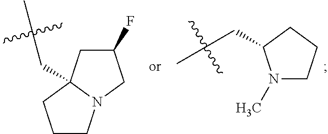

wherein [symbol] represents the point of attachment to X.

In some aspects, R⁵ is

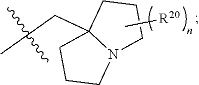

wherein
n is 0, 1, or 2;
each R²⁰ is halo; and
[symbol] represents the point of attachment to X.

In some aspects, Z is a bond.

In some aspects, R¹ is a monocyclic heteroaryl ring containing one, two, or three nitrogen atoms, wherein the ring is optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

In some aspects, R¹ is

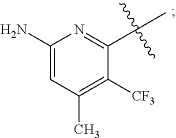

wherein [symbol] denotes the point of attachment to the parent molecular moiety.

In some aspects, R is C₆-C₁₀aryl optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

In some aspects, R¹ is naphtyl substituted with one, two, three, four, or five substituents independently selected from C₁-C₃alkyl, C₂-C₄alkynyl, halo, and hydroxy.

In some aspects, R¹ is naphthyl, wherein the naphthyl is substituted with one, two, or three groups independently selected from C₂-C₄alkynyl, halo, and hydroxy.

In some aspects, R¹ is

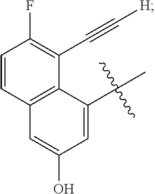

wherein [symbol] denotes the point of attachment to the parent molecular moiety.

In some aspects, R′ is fluoro.

In some aspects, R′ is chloro.

In some aspects, the present disclosure provides a compound of formula (Ia):

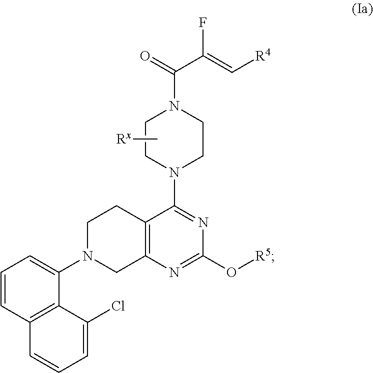

or a pharmaceutically acceptable salt thereof, wherein:
R^{X} is selected from —CH₂CN and methyl;
R⁴ is selected from imidazolyl, isothiazolyl, isoxazolyl, oxazolyl, pyridinyl, pyrimidinyl, thiazolyl, and triazolyl, wherein each ring is optionally substituted with a methyl or halo; and
R⁵ is

[symbol]

In some aspects, the present disclosure provides a compound selected from

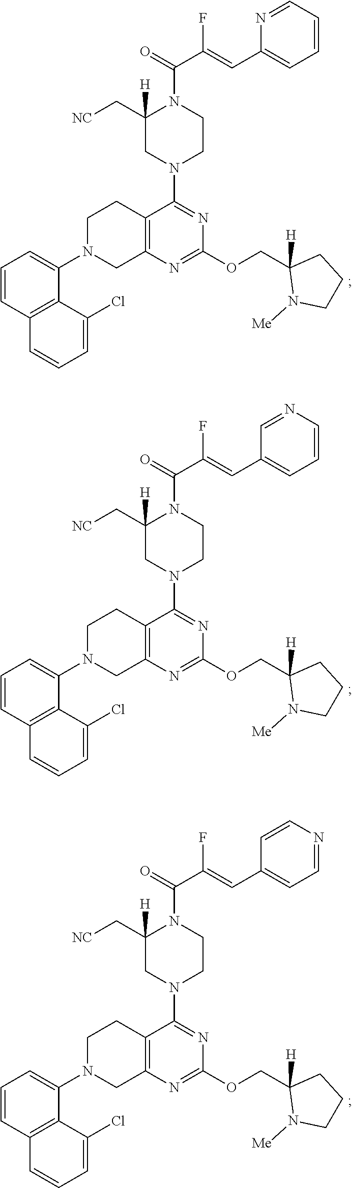 [SMILES: CN1CCC[C@H]1COc1nc2c(c(N3CCN(C(=O)/C(F)=C/c4ccccn4)[C@@H](CC#N)C3)n1)CCN(c1cccc3cccc(Cl)c13)C2 ; CN1CCC[C@H]1COc1nc2c(c(N3CCN(C(=O)/C(F)=C/c4cccnc4)[C@@H](CC#N)C3)n1)CCN(c1cccc3cccc(Cl)c13)C2 ; CN1CCC[C@H]1COc1nc2c(c(N3CCN(C(=O)/C(F)=C/c4ccncc4)[C@@H](CC#N)C3)n1)CCN(c1cccc3cccc(Cl)c13)C2]

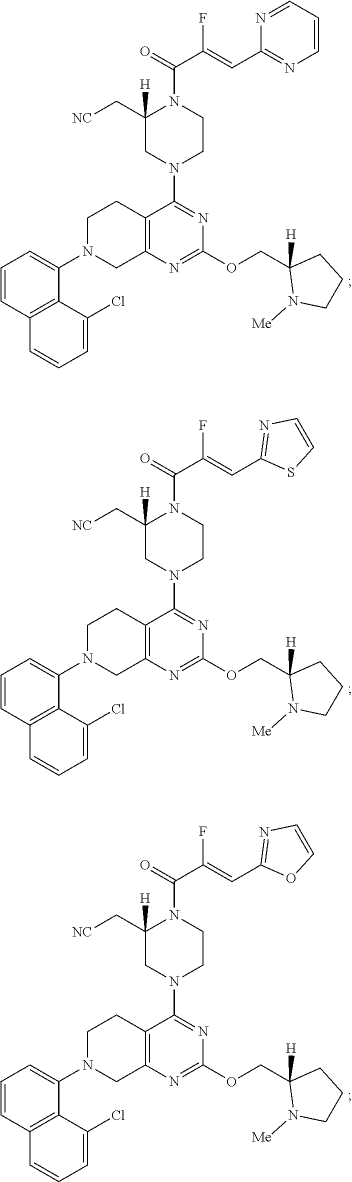 [SMILES: CN1CCC[C@H]1COc1nc2c(c(N3CCN(C(=O)/C(F)=C/c4ncccn4)[C@@H](CC#N)C3)n1)CCN(c1cccc3cccc(Cl)c13)C2 ; CN1CCC[C@H]1COc1nc2c(c(N3CCN(C(=O)/C(F)=C/c4nccs4)[C@@H](CC#N)C3)n1)CCN(c1cccc3cccc(Cl)c13)C2 ; CN1CCC[C@H]1COc1nc2c(c(N3CCN(C(=O)/C(F)=C/c4ncco4)[C@@H](CC#N)C3)n1)CCN(c1cccc3cccc(Cl)c13)C2]

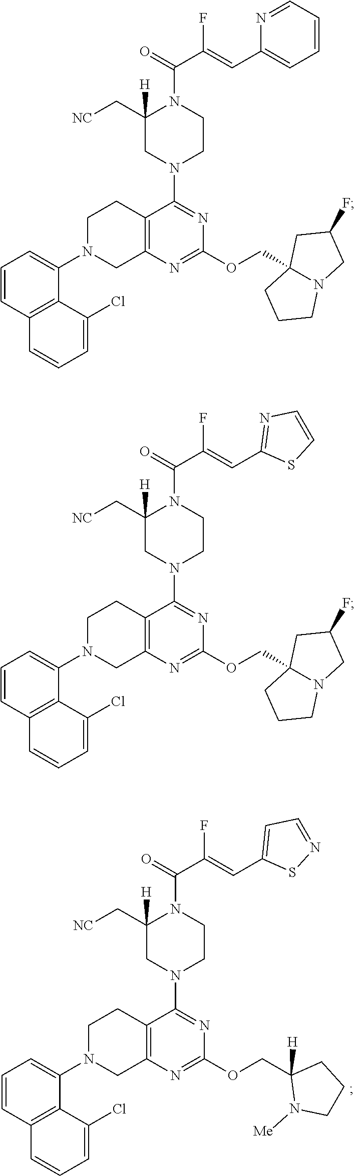 [SMILES: N#CC[C@H]1CN(c2nc(OC[C@@]34CCCN3C[C@H](F)C4)nc3c2CCN(c2cccc4cccc(Cl)c24)C3)CCN1C(=O)/C(F)=C/c1ccccn1 ; N#CC[C@H]1CN(c2nc(OC[C@@]34CCCN3C[C@H](F)C4)nc3c2CCN(c2cccc4cccc(Cl)c24)C3)CCN1C(=O)/C(F)=C/c1nccs1 ; CN1CCC[C@H]1COc1nc2c(c(N3CCN(C(=O)/C(F)=C/c4ccns4)[C@@H](CC#N)C3)n1)CCN(c1cccc3cccc(Cl)c13)C2]

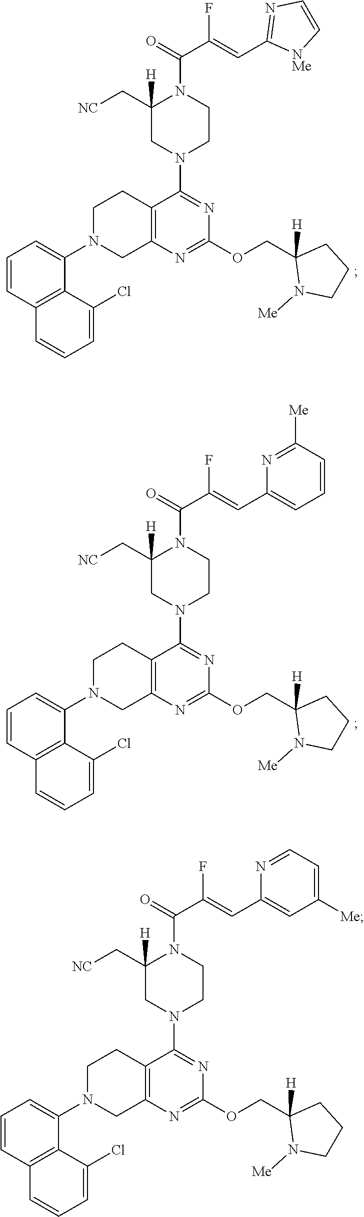 [SMILES: CN1CCC[C@H]1COc1nc2c(c(N3CCN(C(=O)/C(F)=C/c4nccn4C)[C@@H](CC#N)C3)n1)CCN(c1cccc3cccc(Cl)c13)C2 ; Cc1cccc(/C=C(\F)C(=O)N2CCN(c3nc(OC[C@@H]4CCCN4C)nc4c3CCN(c3cccc5cccc(Cl)c35)C4)C[C@@H]2CC#N)n1 ; Cc1ccnc(/C=C(\F)C(=O)N2CCN(c3nc(OC[C@@H]4CCCN4C)nc4c3CCN(c3cccc5cccc(Cl)c35)C4)C[C@@H]2CC#N)c1]

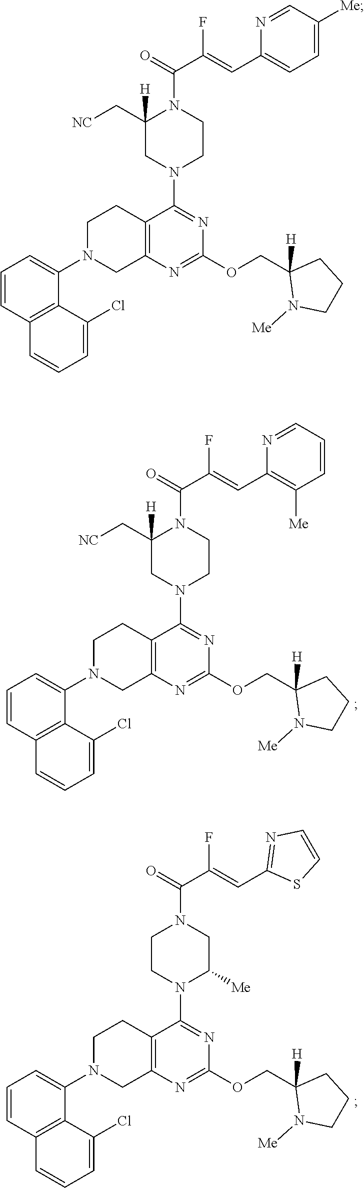 [SMILES: Cc1ccc(/C=C(\F)C(=O)N2CCN(c3nc(OC[C@@H]4CCCN4C)nc4c3CCN(c3cccc5cccc(Cl)c35)C4)C[C@@H]2CC#N)nc1 ; Cc1cccnc1/C=C(\F)C(=O)N1CCN(c2nc(OC[C@@H]3CCCN3C)nc3c2CCN(c2cccc4cccc(Cl)c24)C3)C[C@@H]1CC#N ; C[C@H]1CN(C(=O)/C(F)=C/c2nccs2)CCN1c1nc(OC[C@@H]2CCCN2C)nc2c1CCN(c1cccc3cccc(Cl)c13)C2]

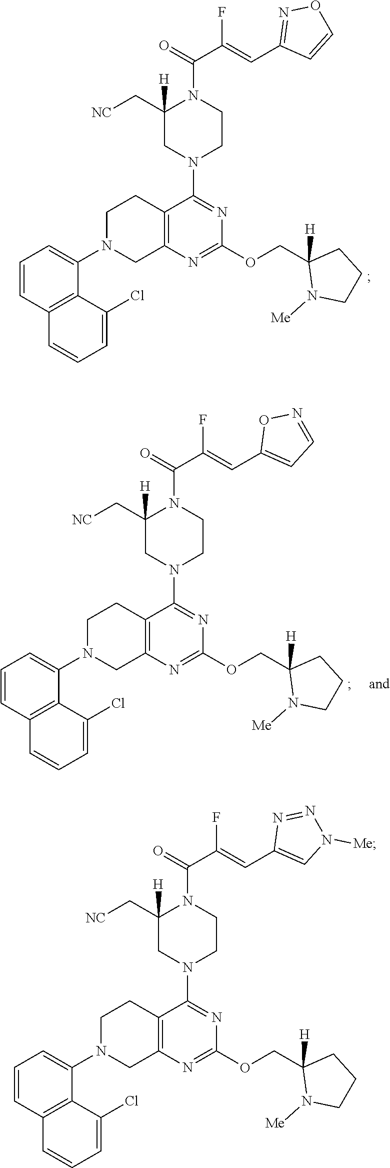 [SMILES: CN1CCC[C@H]1COc1nc2c(c(N3CCN(C(=O)/C(F)=C/c4ccon4)[C@@H](CC#N)C3)n1)CCN(c1cccc3cccc(Cl)c13)C2 ; CN1CCC[C@H]1COc1nc2c(c(N3CCN(C(=O)/C(F)=C/c4ccno4)[C@@H](CC#N)C3)n1)CCN(c1cccc3cccc(Cl)c13)C2 ; CN1CCC[C@H]1COc1nc2c(c(N3CCN(C(=O)/C(F)=C/c4cn(C)nn4)[C@@H](CC#N)C3)n1)CCN(c1cccc3cccc(Cl)c13)C2]

or pharmaceutically acceptable salt thereof.

In some aspects, the present disclosure provides a compound selected from:
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-3-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-4-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyrimidin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(oxazol-2-yl)acryloyl)piperazin-2-yl)acetonitrile;
2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile;
2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoro-3-(isothiazol-5-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(1-methyl-1H-imidazol-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(6-methylpyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(4-methylpyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(5-methylpyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(3-methylpyridin-2-yl)acryloyl)piperazin-2-yl)acetonitrile;
(Z)-1-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-3-methylpiperazin-1-yl)-2-fluoro-3-(thiazol-2-yl)prop-2-en-1-one;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(isoxazol-3-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(isoxazol-5-yl)piperazin-2-yl)acetonitrile; and
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(1-methyl-1H-1,2,3-triazol-4-yl)piperazin-2-yl)acetonitrile;
   or a pharmaceutically acceptable salt thereof.

In some aspects, the present disclosure provides an atropisomer of a compound of any of the prior aspects. In certain aspects, the compound is a stable atropisomer as described herein.

In some aspects, the present disclosure provides a pharmaceutical composition comprising a compound of any of the prior aspects, or a pharmaceutically acceptable salt thereof, and one or more pharmaceutically acceptable excipients.

In some aspects, the present disclosure provides an oral dosage form comprising a compound of any of the prior aspects, or a pharmaceutically acceptable salt thereof, and one or more pharmaceutically acceptable excipients.

In some aspects, the present disclosure provides a method of treating cancer expressing KRAS G12C, G12D and/or G12V mutation in a subject in need thereof, the method comprising administering to the subject a compound of any of the prior aspects, or a pharmaceutically acceptable salt thereof.

In some aspects, the present disclosure provides a method of treating cancer expressing KRAS G12C mutation in a subject in need thereof, the method comprising administering to the subject a compound of any of the prior aspects, or a pharmaceutically acceptable salt thereof.

In some aspects, the present disclosure provides a method for treating a cancer susceptible to KRAS G12C inhibition in a subject in need thereof, the method comprising administering to the subject a compound of any of the prior aspects, or a pharmaceutically acceptable salt thereof.

In some aspects, the present disclosure provides a method for treating a cancer in a subject in need thereof, the method comprising administering to the subject a compound of any of the prior aspects, or a pharmaceutically acceptable salt thereof, wherein the cancer is lung cancer, colorectal cancer, pancreatic cancer, breast cancer, bladder cancer, cervical cancer, ovarian cancer, gastric cancer or cancer of the uterus.

In some aspects, the present disclosure provides a method for treating a cancer in a subject in need thereof, the method comprising administering to the subject a compound of any of the prior aspects, or a pharmaceutically acceptable salt thereof, wherein the cancer is non-small cell lung cancer.

In some aspects of the method, the compound is an atropisomer of a compound of any of the prior aspects. In certain aspects, the compound is a stable atropisomer as described herein.

In another aspect, the present disclosure provides a method for inhibiting KRAS G12C activity in a in a cell, comprising contacting the cell with a therapeutically effective amount of a compound of formula (I), or a pharmaceutically acceptable salt thereof, or a pharmaceutical composition thereof as defined herein. In one aspect, the contacting is in vitro. In one aspect, the contacting is in vivo.

In some aspects, the present disclosure provides a method of inhibiting cell proliferation, in vitro or in vivo, the method comprising contacting a cell with a therapeutically effective amount of a compound of formula (I), or a pharmaceutically acceptable salt thereof, or a pharmaceutical composition thereof as defined herein.

In another aspect, the present disclosure provides a compound of formula (I), or a pharmaceutically acceptable salt thereof, for use in the inhibition of KRAS G12C.

In another aspect, the present disclosure provides a compound of formula (I), or a pharmaceutically acceptable salt thereof, or a pharmaceutical composition thereof as defined herein, for use in the treatment of a KRAS G12C-associated disease or disorder.

In another aspect, the present disclosure provides a use of a compound of formula (I), or a pharmaceutically acceptable salt thereof, as defined herein in the manufacture of a medicament for the treatment of cancer. In some aspects, the cancer is lung cancer. In some aspects, the cancer is non-small cell lung cancer.

In another aspect, the present disclosure provides the use of a compound of formula (I), or a pharmaceutically acceptable salt thereof, as defined herein in the manufacture of a medicament for the inhibition of activity of KRAS G12C.

In another aspect, the present disclosure provides the use of a compound of formula (I), or a pharmaceutically acceptable salt thereof, as defined herein, in the manufacture of a medicament for the treatment of a KRAS G12C-associated disease or disorder.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₂-C₄alkenyl”, as used herein, refers to a group derived from a straight or branched chain hydrocarbon containing from two to four carbon atoms and one double bond.

The term “C₁-C₃alkoxy”, as used herein, refers to a C₁-C₃alkyl group attached to the parent molecular moiety through an oxygen atom.

The term “C₁-C₃alkoxyC₁-C₆alkyl,” as used herein, refers to a C₁-C₃alkoxy group attached to the parent molecular moiety through a C₁-C₆alkyl group.

The term “C₁-C₃alkoxycarbonyl”, as used herein, refers to a C₁-C₃alkoxy group attached to the parent molecular moiety through a carbonyl group.

The term “C₁-C₃alkyl”, as used herein, refers to a group derived from a straight or branched chain saturated hydrocarbon containing from one to three carbon atoms.

The term “C₁-C₃alkylcarbonyl”, as used herein, refers to a C₁-C₃alkyl group attached to the parent molecular moiety through a carbonyl group.

The term “C₂-C₄alkynyl”, as used herein, refers to a group derived from a straight or branched chain hydrocarbon containing from two to four carbon atoms and one triple bond.

The term “amino,” as used herein, refers to —NH₂.

The term “aminoC₁-C₃alkyl,” as used herein, refers to an amino group attached to the parent molecular moiety through a C₁-C₃alkyl group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₆alkyl,” as used herein refers to an aryl group attached to the parent molecular moiety through a C₁-C₆alkyl group.

The term “carboxy,” as used herein, refers to —CO₂H.

The term “carboxyC₁-C₆alkyl,” as used herein, refers to a C₁-C₆alkyl group substituted with one, two, or three carboxy groups.

The term “cyano”, as used herein, refers to —CN.

The term “C₃-C₄cycloalkyl”, as used herein, refers to a saturated monocyclic hydrocarbon ring system having three or four carbon atoms and zero heteroatoms.

The term “C₃-C₆cycloalkyl”, as used herein, refers to a saturated monocyclic hydrocarbon ring system having three to six carbon atoms and zero heteroatoms.

The term “C₃-C₆cycloalkylC₁-C₆alkyl”, as used herein, refers to a C₃-C₆cycloalkyl attached to the parent molecular moiety through a C₁-C₆alkyl group.

The term “di(C₁-C₆alkyl)amino”, as used herein, refers to —NR^{z}R^{z′}, wherein R^{z} and R^{z′} are the same or different C₁-C₆alkyl groups.

The term “di(C₁-C₃alkyl)aminoC₂-C₆alkyl,” as used herein, refers to —(C₂-C₆alkyl)NR^{z}R^{z′}, wherein R^{z} and R^{z′} are the same or different C₁-C₆alkyl groups.

The terms “halo” and “halogen”, as used herein, refer to F, Cl, Br, or I.

The term “haloC₁-C₃alkoxy”, as used herein, refers to a C₁-C₃alkoxy group substituted with one, two, or three halogen atoms.

The term “haloC₁-C₃alkyl”, as used herein, refers to a C₁-C₃alkyl group substituted with one, two, or three halogen atoms.

The term “haloC₁-C₆alkyl”, as used herein, refers to a C₁-C₆alkyl group substituted with one to six halogen atoms.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl, as used herein, refers to a heteroaryl group attached to the parent molecular moiety through a C₁-C₆alkyl group.

The term “heterocyclyl”, as used herein, refers to a five-, six-, seven-, eight-, nine-, ten-, eleven-, or twelve-membered saturated or partially unsaturated ring containing one, two, or three heteroatoms independently selected from nitrogen, oxygen, and sulfur. The term “heterocyclyl” also includes groups in which the heterocyclyl ring is fused to one, two, or three four- to six-membered aromatic or non-aromatic carbocyclic rings or monocyclic heterocyclyl groups. The term “heterocyclyl” also includes monocyclic or polycyclic heterocyclyl group as described above which are further substituted with one or more spirocyclic groups that are attached to the heterocyclyl group through a spiro carbon. Examples of heterocyclyl groups include, but are not limited to, dihydro-1′H,3′H,5′H-dispiro[cyclopropane-1,2′-pyrrolizine-6′,1″-cyclopropane], hexahydro-2H-1,4-dioxa-2al-azacyclopenta[cd]pentalenyl, hexahydropyrrolizinyl, indolinyl, morpholinyl, octahydroindolizinyl, octahydroquinolizinyl, piperazinyl, piperidinyl, pyrrolidinyl, tetrahydrofuranyl, tetrahydropyranyl, and thiomorpholinyl.

The term “heterocyclylC₁-C₆alkyl, as used herein, refers to a heterocyclyl group attached to the parent molecular moiety through a C₁-C₆alkyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₆alkyl,” as used herein, refers to a hydroxy group attached to the parent molecular moiety through a C₁-C₆alkyl group.

The term “NR^{a}R^{b}—C(O),” as used herein, refers to an NR^{a}R^{b} group attached to the parent molecular moiety through a carbonyl group.

The term “NR^{a}R^{b}—C(O)—C₁-C₆alkyl,” as used herein, refers to an NR^{a}R^{b}—C(O)— group attached to the parent molecular moiety through a C₁-C₆alkyl group.

The term “NR^{a}R^{b}C₁-C₆alkyl, as used herein, refers to an NR^{a}R^{b} group attached to the parent molecular moiety through a C₁-C₆alkyl group.

The term “nitro,” as used herein, refers to —NO₂.

The term “oxo,” as used herein, refers to ═O.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a compound described herein can be prepared using a radioactive isotope and the resulting radiolabeled compound can be used to develop a binding assay or for metabolism studies. Alternatively, and for the same purpose, a compound described herein can be converted to a radiolabeled form by catalytic tritiation using methods known to those skilled in the art.

Certain compounds of the present disclosure exist as stereoisomers. It should be understood that when stereochemistry is not specified, the present disclosure encompasses all stereochemical isomeric forms, or mixtures thereof, which possess the ability inhibit KRAS G12C. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure exist as atropisomers. The term “atropisomers” refers to conformational stereoisomers which occur when rotation about a single bond in the molecule is prevented, or greatly slowed, as a result of steric interactions with other parts of the molecule and the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom.

The atropisomers can be considered stable if the barrier to interconversion is high enough to permit the atropisomers to undergo little or no interconversion at room temperature for at least a week. In some aspects the atropisomers undergo little or no interconversion at room temperature for at least a year. In some aspects, an atropisomeric compound of the disclosure does not undergo more than about 5% interconversion to its opposite atropisomer at room temperature during one week when the atropisomeric compound is in substantially pure form, which is generally a solid state. In some aspects, an atropisomeric compound of the disclosure does not undergo more than about 5% interconversion to its opposite atropisomer at room temperature (approximately 25° C.) during one year. In some aspects, the atropisomeric compounds of the disclosure are stable enough to undergo no more than about 5% interconversion in an aqueous pharmaceutical formulation held at 0° C. for at least one week. The present chemical entities, pharmaceutical compositions and methods are meant to include all such possible atropisomers, including racemic mixtures, diastereomeric mixtures, epimeric mixtures, optically pure forms of single atropisomers, and intermediate mixtures.

The energy barrier to thermal racemization of atropisomers may be determined by the steric hindrance to free rotation of one or more bonds forming a chiral axis. Certain biaryl compounds exhibit atropisomerism where rotation around an interannular bond lacking C₂ symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biaryl compounds may exhibit this type of conformational, rotational isomerism. Such biaryls are enantiomeric, chiral atropisomers where the sp²-sp² carbon-carbon, interannular bond between the aryl rings has a sufficiently high energy barrier to prevent free rotation, and where substituents W¹≠W² and W³≠W⁴ render the molecule asymmetric.

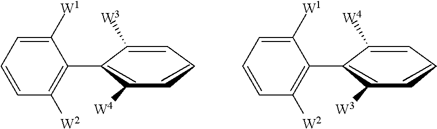

The steric interaction between W¹:W³, W¹:W⁴, and/or W²:W⁴, W²:W³ is large enough to make the planar conformation an energy maximum. Two non-planar, axially chiral enantiomers then exist as atropisomers when their interconversion is slow enough such that they can be isolated free of each other. Bold lines and dashed lines in the figures shown above indicate those moieties, or portions of the molecule, which are sterically restricted due to a rotational energy barrier. Balded moieties exist orthogonally above the plane of the page, and dashed moieties exist orthogonally below the plane of the page. The ‘flat’ part of the molecule (the left ring in each of the two depicted biaryls) is in the plane of the page.

The pharmaceutical compositions of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents, as described herein.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

The pharmaceutical compositions of the present disclosure can be administered via one or more routes of administration using one or more of a variety of methods known in the art. As will be appreciated by the skilled artisan, the route and/or mode of administration will vary depending upon the desired results. In some aspects, the routes of administration for compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are reduced pressure drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, and injectable organic esters. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution or as a liquid with ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets.

An aqueous suspension can be prepared, for example, by admixing at least one compound of Formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one aspect, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain aspects, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some aspects, the compounds of the present disclosure can be administered orally, i.e, via a gelatin capsule, tablet, hard or soft capsule, or a liquid capsule.

### Use of KRAS Inhibitors/Methods of Treating

Administration of a therapeutic agent described herein may include administration of a therapeutically effective amount of therapeutic agent. The term “therapeutically effective amount” as used herein refers, without limitation, to an amount of a therapeutic agent to treat a condition treatable by administration of a composition comprising the KRAS inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the compounds described herein, the dosage ranges from about 0.0001 to 100 mg/kg, and more usually 0.01 to 40 mg/kg, of the host body weight. An exemplary treatment regime entails administration once per day, bi-weekly, tri-weekly, weekly, once every two weeks, once every three weeks, once every four weeks, once a month, once every 3 months or once every three to 6 months.

The disclosed compounds strongly inhibit anchorage-independent cell growth and therefore have the potential to inhibit tumor metastasis. Accordingly, in another aspect the disclosure provides a method for inhibiting tumor metastasis, the method comprising administering an effective amount a pharmaceutical composition of comprising any of the compounds disclosed herein and a pharmaceutically acceptable carrier to a subject in need thereof.

Ras mutations including but not limited to KRAS mutations have also been identified in hematological malignancies (e.g., cancers that affect blood, bone marrow and/or lymph nodes). Accordingly, certain aspects are directed to administration of a disclosed compounds (e.g., in the form of a pharmaceutical composition) to a patient in need of treatment of a hematological malignancy. Such malignancies include, but are not limited to leukemias and lymphomas. For example, the presently disclosed compounds can be used for treatment of diseases such as Acute lymphoblastic leukemia (ALL), Acute myelogenous leukemia (AML), Chronic lymphocytic leukemia (CLL), small lymphocytic lymphoma (SLL), Chronic myelogenous leukemia (CML), Acute monocytic leukemia (AMoL) and/or other leukemias. In other aspects, the compounds are useful for treatment of lymphomas such as all subtypes of Hodgkins lymphoma or non-Hodgkins lymphoma.

Determining whether a tumor or cancer comprises a KRAS mutation can be undertaken by assessing the nucleotide sequence encoding the KRAS protein, by assessing the amino acid sequence of KRAS protein, or by assessing the characteristics of a putative KRAS mutant protein. The sequence of wild-type human KRAS proteins is known in the art.

Methods for detecting a KRAS mutation are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some aspects, samples are evaluated for KRAS mutations including by real-time PCR. In real-time PCR, fluorescent probes specific for the KRAS mutation are used. When a mutation is present, the probe binds and fluorescence is detected. In some aspects, the KRAS mutation is identified using a direct sequencing method of specific regions (e.g., exon 2 and/or exon 3) in the KRAS gene, for example. This technique will identify all possible mutations in the region sequenced.

Methods for detecting a mutation in a KRAS protein are known by those of skill in the art. These methods include, but are not limited to, detection of a KRAS mutant using a binding agent (e.g., an antibody) specific for the mutant protein, protein electrophoresis and Western blotting, and direct peptide sequencing.

Methods for determining whether a tumor or cancer comprises a KRAS mutation can use a variety of samples. In some aspects, the sample is taken from a subject having a tumor or cancer. In some aspects, the sample is taken from a subject having a cancer or tumor. In some aspects, the sample is a fresh tumor/cancer sample. In some aspects, the sample is a frozen tumor/cancer sample. In some aspects, the sample is a formalin-fixed paraffin-embedded sample. In some aspects, the sample is processed to a cell lysate. In some aspects, the sample is processed to DNA or RNA. The disclosure also relates to a method of treating a hyperproliferative disorder in a mammal that comprises administering to said mammal a therapeutically effective amount of a compound of the present disclosure, or a pharmaceutically acceptable salt, ester, prodrug, solvate, hydrate or derivative thereof. In some aspects, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, isletcell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects, said method relates to the treatment of a non-cancerous hyperproliferative disorder such as benign hyperplasia of the skin (e.g., psoriasis), restenosis, or prostate (e.g., benign prostatic hypertrophy (BPH)).

In certain aspects, the disclosure relates to methods for treatment of lung cancers, the methods comprise administering an effective amount of any of the above-described compound (or a pharmaceutical composition comprising the same) to a subject in need thereof. In certain aspects the lung cancer is a non-small cell lung carcinoma (NSCLC), for example adenocarcinoma, squamous-cell lung carcinoma or large-cell lung carcinoma. In other aspects, the lung cancer is a small cell lung carcinoma. Other lung cancers treatable with the disclosed compounds include, but are not limited to, glandular tumors, carcinoid tumors and undifferentiated carcinomas. Subjects that can be treated with compounds of the disclosure, or pharmaceutically acceptable salt, ester, prodrug, solvate, tautomer, hydrate or derivative of said compounds, according to the methods of this disclosure include, for example, subjects that have been diagnosed as having acute myeloid leukemia, acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germcell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasalcavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects subjects that are treated with the compounds of the disclosure include subjects that have been diagnosed as having a non-cancerous hyperproliferative disorder such as benign hyperplasia of the skin (e. g., psoriasis), restenosis, or prostate (e. g., benign pro static hypertrophy (BPH)). The disclosure further provides methods of modulating a mutant KRAS protein activity by contacting the protein with an effective amount of a compound of the disclosure. Modulation can be inhibiting or activating protein activity. In some aspects, the disclosure provides methods of inhibiting protein activity by contacting the mutant KRAS protein with an effective amount of a compound of the disclosure in solution. In some aspects, the disclosure provides methods of inhibiting the mutant KRAS protein activity by contacting a cell, tissue, organ that express the protein of interest. In some aspects, the disclosure provides methods of inhibiting protein activity in a subject including but not limited to rodents and mammal (e.g., human) by administering into the subject an effective amount of a compound of the disclosure. In some aspects, the percentage modulation exceeds 25%, 30%, 40%, 50%, 60%, 70%, 80%, or 90%. In some aspects, the percentage of inhibiting exceeds 25%, 30%, 40%, 50%, 60%, 70%, 80%, or 90%. In some aspects, the disclosure provides methods of inhibiting KRAS activity in a cell by contacting said cell with an amount of a compound of the disclosure sufficient to inhibit the activity of a KRAS mutant in said cell. In some aspects, the disclosure provides methods of inhibiting mutant KRAS in a tissue by contacting said tissue with an amount of a compound of the disclosure sufficient to inhibit the activity of mutant KRAS in said tissue. In some aspects, the disclosure provides methods of inhibiting KRAS in an organism by contacting said organism with an amount of a compound of the disclosure sufficient to inhibit the activity of KRAS in said organism. In some aspects, the disclosure provides methods of inhibiting KRAS activity in an animal by contacting said animal with an amount of a compound of the disclosure sufficient to inhibit the activity of KRAS in said animal. In some aspects, the disclosure provides methods of inhibiting KRAS including in a mammal by contacting said mammal with an amount of a compound of the disclosure sufficient to inhibit the activity of KRAS in said mammal. In some aspects, the disclosure provides methods of inhibiting KRAS activity in a human by contacting said human with an amount of a compound of the disclosure sufficient to inhibit the activity of KRAS in said human. The present disclosure provides methods of treating a disease mediated by KRAS activity in a subject in need of such treatment. The present disclosure also provides methods for combination therapies in which an agent known to modulate other pathways, or other components of the same pathway, or even overlapping sets of target enzymes are used in combination with a compound of the present disclosure, or a pharmaceutically acceptable salt, ester, prodrug, solvate, tautomer, hydrate or derivative thereof. In one aspect, such therapy includes but is not limited to the combination of one or more compounds of the disclosure with chemotherapeutic agents, therapeutic antibodies, and radiation treatment.

Many chemotherapeutics are presently known in the art and can be used in combination with the compounds of the disclosure. In some aspects, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

The compounds described herein can be used in combination with the agents disclosed herein or other suitable agents, depending on the condition being treated. Hence, in some aspects, the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some aspects of the separate administration protocol, a compound of the disclosure and any of the agents described above are administered a few minutes apart, or a few hours apart, or a few days apart.

The compounds can be made by methods known in the art including those described below and including variations within the skill of the art. Some reagents and intermediates are known in the art. Other reagents and intermediates can be made by methods known in the art using readily available materials. Any variables (e.g., numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### Synthesis

The aspects described herein are further defined in the following Examples. It should be understood that the Examples are given by way of illustration only. From the above discussion and the Examples, one skilled in the art can ascertain the essential characteristics of the aspects described herein, and without departing from the spirit and scope thereof, can make various changes and modifications to adapt them to various uses and conditions. As a result, the aspects described herein are not limited by the illustrative examples set forth herein below, but rather are defined by the claims appended hereto.

### Abbreviations

The following abbreviations are used in the example section below and elsewhere herein: DIPEA for diisopropylethylaminde; DMF for N,N-dimethylformamide; DMSO for dimethylsulfoxide; MeCN or ACN for acetonitrile; TFA for trifluoroacetic acid;

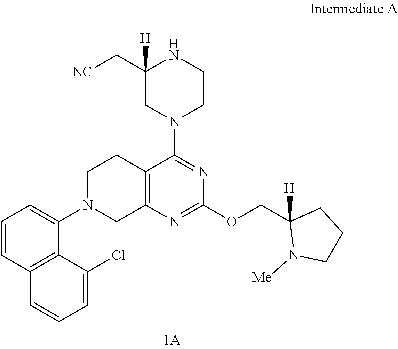 [SMILES: CN1CCC[C@H]1COc1nc2c(c(N3CCN[C@@H](CC#N)C3)n1)CCN(c1cccc3cccc(Cl)c13)C2]

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile;

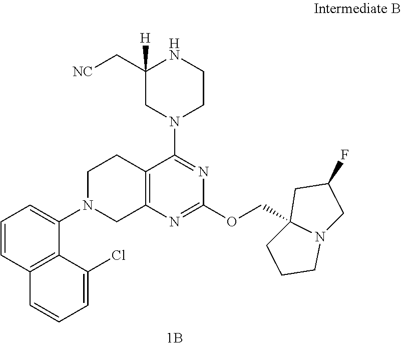 [SMILES: N#CC[C@H]1CN(c2nc(OC[C@@]34CCCN3C[C@H](F)C4)nc3c2CCN(c2cccc4cccc(Cl)c24)C3)CCN1]

2-((S)-4-(7-(8-chloronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperazin-2-yl)acetonitrile); and

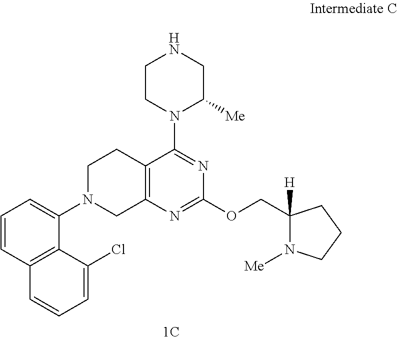 [SMILES: C[C@H]1CNCCN1c1nc(OC[C@@H]2CCCN2C)nc2c1CCN(c1cccc3cccc(Cl)c13)C2]

All three compounds were prepared according to the synthetic procedures described in U.S. Pat. No. 10,689,377 B2.

### Preparation of Heteroaryl Intermediates

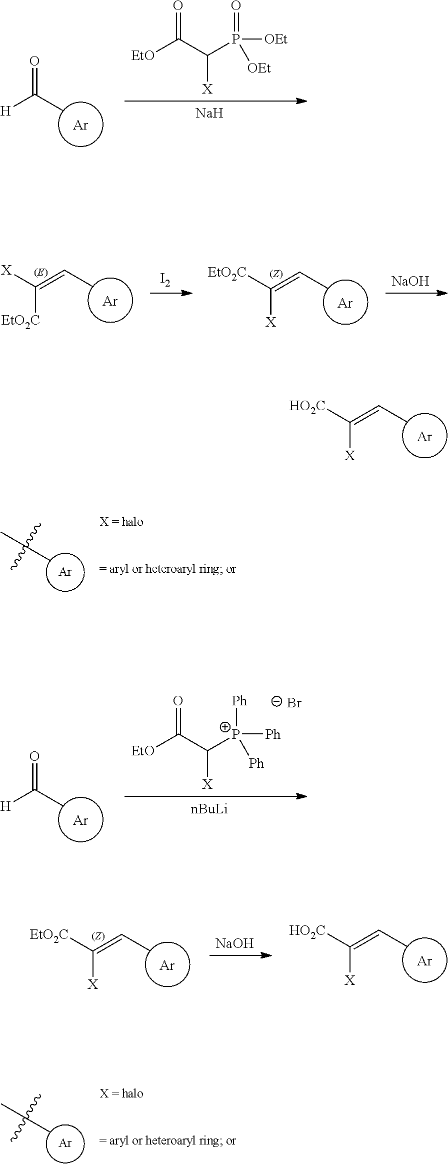

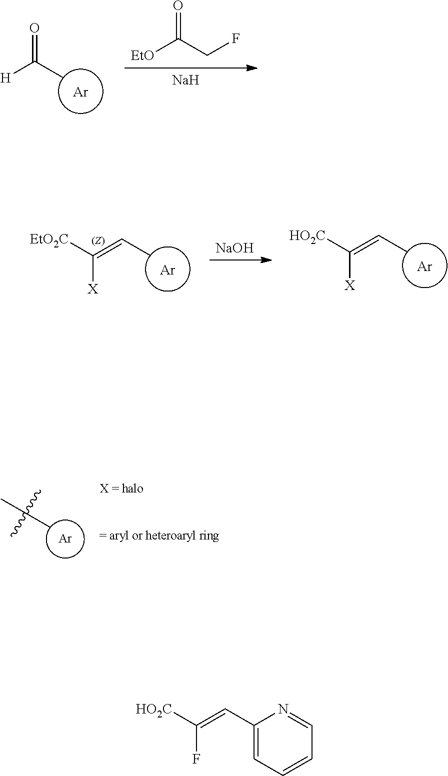

### Intermediate D

### (Z)-2-fluoro-3-(pyridin-2-yl)acrylic acid

### Step 1: Preparation of ethyl (E)-2-fluoro-3-(pyridin-2-yl)acrylate

Ethyl 2-(diethoxyphosphoryl)-2-fluoroacetate (1.5 g, 6.2 mmol) was dissolved in THE (31 mL). The solution was cooled to 0° C. Sodium hydride (60% dispersion in mineral oil, 0.25 g, 6.2 mmol) was added portionwise as a solid. The reaction mixture was stirred for 10 min and picolinaldehyde (0.66 g, 6.2 mmol) was added. The reaction mixture was allowed to warm to room temperature and was stirred for 1 h. The mixture was quenched by addition of saturated aqueous ammonium chloride solution (15 mL). The solution was diluted with water (20 mL) and EtOAc (100 mL). The layers were separated and the aqueous phase was further extracted with EtOAc (2×50 mL). The combined organic layers were dried over magnesium sulfate, filtered, and concentrated. The crude residue was purified by column chromatography (0→100% EtOAc/hexanes) to afford ethyl (E)-2-fluoro-3-(pyridin-2-yl)acrylate (3:1 E/Z mixture as judged by ¹H NMR, 910 mg, 4.6 mmol, 75% yield). LC/MS (ESI) m/z: [M+H]⁺ calcd for C₁₀H₁₁FNO₂ 196.1; found 196.1; E isomer reported: ¹H NMR (500 MHz, CDCl₃) δ 8.59 (dd, J=4.9, 1.8 Hz, 1H), 7.67 (ddd, J=7.9, 7.6, 1.8 Hz, 1H), 7.56 (d, J=7.9 Hz, 1H), 7.21 (dd, J=7.6, 4.9 Hz, 1H), 6.90 (d, J=20.4 Hz, 1H), 4.25 (q, J=7.2 Hz, 2H), 1.23 (t, J=7.1 Hz, 3H).

### Step 2: Preparation of ethyl (Z)-2-fluoro-3-(pyridin-2-yl)acrylate

Ethyl (E)-2-fluoro-3-(pyridin-2-yl)acrylate (420 mg, 2.1 mmol) was dissolved in toluene (10 mL) and iodine (27 mg, 0.15 mmol) was added. The reaction mixture was heated at 100° C. for 7 days. The solution was concentrated and purified by column chromatography (0→100% EtOAc/hexanes) to afford ethyl (Z)-2-fluoro-3-(pyridin-2-yl)acrylate (240 mg, 1.2 mmol, 58% yield). LC/MS (ESI) m/z: [M+H]⁺ calcd for C₁₀H₁₁FNO₂ 196.1; found 196.1; ¹H NMR (500 MHz, CDCl₃) δ 8.66 (ddd, J=5.0, 1.8, 0.8 Hz, 1H), 7.88 (ddd, J=8.0, 1.2, 0.8 Hz, 1H), 7.75 (ddd, J=8.0, 7.8, 1.8 Hz, 1H), 7.25 (ddd, J=7.8, 5.0, 1.2 Hz, 1H), 7.14 (d, J=34.9 Hz, 1H), 4.36 (q, J=7.2 Hz, 2H), 1.38 (t, J=7.2 Hz, 3H).

### Step 3: Preparation of (Z)-2-fluoro-3-(pyridin-2-yl)acrylic acid

Ethyl (Z)-2-fluoro-3-(pyridin-2-yl)acrylate (415 mg, 2.1 mmol) was dissolved in MeOH (15 mL). The solution was cooled to 0° C. and sodium hydroxide solution (1.0 M, 2.1 mL, 2.1 mmol) was added. The reaction mixture was stirred for 5 h. The solution was concentrated to remove the methanol. Additional water (2.0 mL) was added and the aqueous solution was cooled to 0° C. HCl solution (1.0 M, 2.1 mL, 2.1 mmol) was added dropwise. After 10 min, a white solid precipitated. The solid was collected by filtration and was washed with Et₂O. The solid was dried under vacuum to afford (Z)-2-fluoro-3-(pyridin-2-yl)acrylic acid (235 mg, 1.4 mmol, 66% yield) as a white solid. LC/MS (ESI) m/z: [M+H]⁺ calcd for C₈H₇FNO₂ 168.0; found 167.8. ¹H NMR (500 MHz, DMSO-d₆) δ 8.66 (ddd, J=4.8, 1.8, 0.8 Hz, 1H), 7.89 (ddd, J=8.0, 7.5, 1.8 Hz, 1H), 7.81 (ddd, J=8.0, 1.1, 0.8 Hz, 1H), 7.40 (dd, J=7.5, 4.8, 1.1 Hz, 1H), 6.97 (d, J=35.3 Hz, 1H).

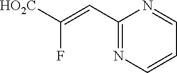 [SMILES: O=C(O)/C(F)=C/c1ncccn1]

### Intermediate E

### (Z)-2-fluoro-3-(pyrimidin-2-yl)acrylic acid

### Step 1: Preparation of ethyl (E)-2-fluoro-3-(pyrimidin-2-yl)acrylate

Ethyl 2-(diethoxyphosphoryl)-2-fluoroacetate (1.6 g, 6.6 mmol) was dissolved in THE (50 mL). The solution was cooled to 0° C. Sodium hydride (60% dispersion in mineral oil, 0.26 g, 6.6 mmol) was added portionwise as a solid. The reaction mixture was stirred for 10 min and pyrimidine-2-carbaldehyde (0.71 g, 6.6 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (150 mL). The layers were separated and the aqueous phase was further extracted with EtOAc (2×50 mL). The combined organic layers were dried over magnesium sulfate, filtered, and concentrated. The crude residue was purified by column chromatography (0→90% EtOAc/hexanes) to afford ethyl (E)-2-fluoro-3-(pyrimidin-2-yl)acrylate (5:1 E/Z as judged by 1H NMR, 592 mg, 3.0 mmol, 46% yield). LC/MS (ESI) m/z: [M+H]⁺ calcd for C₉H₁₀FN₂O₂ 197.2; found 197.1; ¹H NMR (500 MHz, CDCl₃) δ 8.70 (d, J=4.9 Hz, 2H), 7.18 (t, J=4.9 Hz, 1H), 6.77 (d, J=17.5 Hz, 1H), 4.28 (q, J=7.2 Hz, 3H), 1.39 (t, J=7.2 Hz, 3H).

### Step 2: Preparation of ethyl (Z)-2-fluoro-3-(pyrimidin-2-yl)acrylate

Ethyl (E)-2-fluoro-3-(pyrimidin-2-yl)acrylate (592 mg, 3.0 mmol) was dissolved in toluene (15 mL) and iodine (38 mg, 0.15 mmol) was added. The reaction mixture was heated at 100° C. for 7 days. The solution was concentrated and purified by column chromatography (0→100% EtOAc/hexanes) to afford ethyl (Z)-2-fluoro-3-(pyrimidin-2-yl)acrylate (253 mg, 1.3 mmol, 43% yield). LC/MS (ESI) m/z: [M+H]⁺ calcd for C₉H₁₀FN₂O₂ 197.2; found 196.6; ¹H NMR (500 MHz, CDCl₃) δ 8.83 (d, J=4.9 Hz, 2H), 7.21 (t, J=4.9 Hz, 1H), 7.13 (d, J=30.8 Hz, 1H), 4.38 (q, J=7.2 Hz, 2H), 1.39 (t, J=7.2 Hz, 3H).

### Step 3: Preparation of (Z)-2-fluoro-3-(pyrimidin-2-yl)acrylic acid

Ethyl (Z)-2-fluoro-3-(pyrimidin-2-yl)acrylate (253 mg, 1.3 mmol) was dissolved in MeOH (10 mL). The solution was cooled to 0° C. and sodium hydroxide solution (1.0 M, 1.3 mL, 1.3 mmol) was added. The reaction mixture was stirred for 2 h. The solution was concentrated to remove the methanol. Additional water (1.0 mL) was added and the aqueous solution was cooled to 0° C. HCl solution (1.0 M, 2.5 mL, 2.5 mmol) was added dropwise. After 10 min, a white solid precipitated. The solid was collected by filtration and dried under vacuum to afford (Z)-2-fluoro-3-(pyrimidin-2-yl)acrylic acid (190 mg, 1.1 mmol, 88% yield) as a white solid. LC/MS (ESI) m/z: [M+H]⁺ calcd for C₇H₆FN₂O₂ 169.1; found 168.8; ¹H NMR (500 MHz, DMSO-d₆) δ 8.90 (d, J=4.9 Hz, 2H), 7.46 (t, J=4.9 Hz, 1H), 6.94 (d, J=31.5 Hz, 1H).

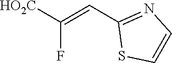 [SMILES: O=C(O)/C(F)=C/c1nccs1]

### Intermediate F

### (Z)-2-fluoro-3-(thiazol-2-yl)acrylic acid

### Step 1: Preparation of ethyl (E)-2-fluoro-3-(thiazol-2-yl)acrylate

Ethyl 2-(diethoxyphosphoryl)-2-fluoroacetate (1.1 g, 4.5 mmol) was dissolved in THE (25 mL). The solution was cooled to 0° C. Sodium hydride (60% dispersion in mineral oil, 0.18 g, 4.5 mmol) was added portionwise as a solid. The reaction mixture was stirred for 10 min and thiazole-2-carbaldehyde (0.51 g, 4.5 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (150 mL). The layers were separated and the aqueous phase was further extracted with EtOAc (2×50 mL). The combined organic layers were dried over magnesium sulfate, filtered, and concentrated. The crude residue was purified by column chromatography (0→60% acetone/hexanes) to afford ethyl (E)-2-fluoro-3-(thiazol-2-yl)acrylate (10:1 E/Z as judged by ¹H NMR, 620 mg, 3.1 mmol, 68% yield). LC/MS (ESI) m/z: [M+H]⁺ calcd for C₈H₉FNO₂S 202.0; found 202.2; ¹H NMR (500 MHz, CDCl₃) δ 7.94 (d, J=3.2 Hz, 1H), 7.52 (d, J=3.2 Hz, 1H), 7.34 (d, J=22.2 Hz, 1H), 4.43 (q, J=7.2 Hz, 2H), 1.41 (t, J=7.2 Hz, 3H).

### Step 2: Preparation of ethyl (Z)-2-fluoro-3-(thiazol-2-yl)acrylate

Ethyl (E)-2-fluoro-3-(thiazol-2-yl)acrylate (620 mg, 3.1 mmol) was dissolved in toluene (15 mL) and iodine (39 mg, 0.15 mmol) was added. The reaction mixture was heated at 100° C. for 7 days. The solution was concentrated and purified by column chromatography (0→100% EtOAc/hexanes) to afford ethyl (Z)-2-fluoro-3-(thiazol-2-yl)acrylate (509 mg, 2.5 mmol, 82% yield). LC/MS (ESI) m/z: [M+H]⁺ calcd for C₈H₉FNO₂S 202.0; found 202.0; ¹H NMR (500 MHz, CDCl₃) δ 7.96 (dd, J=3.2, 2.6 Hz, 1H), 7.57 (d, J=3.2 Hz, 1H), 7.43 (dd, J=33.3, 0.8 Hz, 1H), 4.38 (q, J=7.2 Hz, 2H), 1.39 (t, J=7.1 Hz, 3H).

### Step 3: Preparation of (Z)-2-fluoro-3-(thiazol-2-yl)acrylic acid

Ethyl (Z)-2-fluoro-3-(thiazol-2-yl)acrylate (510 mg, 2.5 mmol) was dissolved in MeOH (15 mL). The solution was cooled to 0° C. and sodium hydroxide solution (1.0 M, 2.5 mL, 2.5 mmol) was added. The reaction mixture was stirred for 5 h. The solution was concentrated to remove the methanol. Additional water (1.5 mL) was added and the aqueous solution was cooled to 0° C. HCl solution (1.0 M, 2.5 mL, 2.5 mmol) was added dropwise. After 10 min, a white solid precipitated. The solid was collected by filtration and was washed with MeCN. The solid was dried under vacuum to afford (Z)-2-fluoro-3-(thiazol-2-yl)acrylic acid (337 mg, 1.9 mmol, 77% yield) as a white solid. LC/MS (ESI) m/z: [M+H]⁺ calcd for C₆H₅FNO₂S 174.0; found 173.8; ¹H NMR (500 MHz, DMSO-d₆) δ 8.04 (s, 2H), 7.29 (d, J=34.5 Hz, 1H).

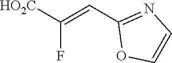 [SMILES: O=C(O)/C(F)=C/c1ncco1]

### Intermediate G

### (Z)-2-fluoro-3-(oxazol-2-yl)acrylic acid

### Step 1: Preparation of ethyl (E)-2-fluoro-3-(oxazol-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and oxazole-2-carbaldehyde (100 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by column chromatography (0→100% EtOAc/hexanes) to afford ethyl (E)-2-fluoro-3-(oxazol-2-yl)acrylate (6:1 E/Z as judged by 1H NMR, 165 mg, 0.89 mmol, 86% yield). LC/MS (ESI) m/z: [M+H]⁺ calcd for C₈H₉FNO₃ 186.0; found 186.0; E isomer reported: ¹H NMR (500 MHz, CDCl₃) δ 7.69 (d, J=0.7 Hz, 1H), 7.25 (d, J=0.7 Hz, 1H), 6.62 (d, J=17.6 Hz, 1H), 4.35 (q, J=7.2 Hz, 2H), 1.32 (t, J=7.2 Hz, 3H).

### Step 2: Preparation of ethyl (Z)-2-fluoro-3-(oxazol-2-yl)acrylate

Ethyl (E)-2-fluoro-3-(oxazol-2-yl)acrylate (165 mg, 0.89 mmol) was dissolved in toluene (2 mL) and iodine (11 mg, 0.045 mmol) was added. The reaction mixture was heated at 100° C. for 7 days. The solution was concentrated and purified by column chromatography (0→100% EtOAc/hexanes) to afford ethyl (Z)-2-fluoro-3-(oxazol-2-yl)acrylate (22 mg, 0.12 mmol, 13% yield). LC/MS (ESI) m/z: [M+H]⁺ calcd for C₈H₉FNO₃ 186.0; found 185.9 ¹H NMR (500 MHz, CDCl₃) δ 7.77 (s, 1H), 7.32 (s, 1H), 6.96 (d, J=31.0 Hz, 1H), 4.37 (q, J=7.2 Hz, 2H), 1.38 (t, J=7.2 Hz, 3H).

### Step 3: Preparation of (Z)-2-fluoro-3-(oxazol-2-yl)acrylic acid

Ethyl (Z)-2-fluoro-3-(oxazol-2-yl)acrylate (22 mg, 0.12 mmol) was dissolved in MeOH (1 mL). The solution was cooled to 0° C. and sodium hydroxide solution (1.0 M, 0.12 mL, 0.12 mmol) was added. The reaction mixture was stirred for 4 h. The solution was concentrated to remove the methanol. Additional water (0.3 mL) was added and the aqueous solution was cooled to 0° C. HCl solution (1.0 M, 0.12 mL, 0.12 mmol) was added dropwise. The solution was frozen and directly lyophilized to afford (Z)-2-fluoro-3-(oxazol-2-yl)acrylic acid (18 mg, 0.12 mmol, quantitative yield assumed; contains 1 equiv NaCl) as a white solid. LC/MS (ESI) m/z: [M+H]⁺ calcd for C₆H₅FNO₃ 158.0; found 157.8; ¹H NMR (600 MHz, DMSO-d₆) δ 8.30 (s, 1H), 7.47 (s, 1H), 6.90 (d, J=32.2 Hz, 1H).

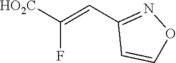 [SMILES: O=C(O)/C(F)=C/c1ccon1]

### Intermediate H

### (Z)-2-fluoro-3-(isoxazol-3-yl)acrylic acid

### Step 1: Preparation of ethyl (Z)-2-fluoro-3-(isoxazol-3-yl)acrylate

(2-ethoxy-1-fluoro-2-oxoethyl)triphenylphosphonium bromide (922 mg, 2.06 mmol) was stirred in THE (10 mL) at 0° C. To the mixture was added dropwise nBuLi solution (2.5 M in hexanes, (0.82 mL, 2.06 mmol) The mixture was stirred for 10 minutes and then isoxazole-3-carbaldehyde (100 mg, 1.030 mmol) was added as a solid. The reaction mixture was allowed to warm to room temperature over two hours. The reaction mixture was concentrated, and the residue was directly purified by column chromatography (5→100% EtOAc/hexanes) to afford ethyl (Z)-2-fluoro-3-(isoxazol-3-yl)acrylate (21 mg, 0.11 mmol, 11% yield). LC/MS (ESI) m/z: [M+H]⁺ calcd for C₈H₉FNO₃ 186.1; found 185.9; ¹H NMR (500 MHz, CDCl₃) δ 8.47 (dd, J=1.7, 0.8 Hz, 1H), 7.16 (d, J=33.0 Hz, 1H), 6.78 (dd, J=2.9, 1.7 Hz, 1H), 4.38 (q, J=7.2 Hz, 2H), 1.38 (t, J=7.2 Hz, 3H).

### Step 2: Preparation of (Z)-2-fluoro-3-(isoxazol-3-yl)acrylic acid

Ethyl (Z)-2-fluoro-3-(isoxazol-3-yl)acrylate (21 mg, 0.11 mmol) was dissolved in MeOH (1.1 mL) and sodium hydroxide solution (1.0 M, 160 μL, 0.16 mmol) was added dropwise. The mixture was stirred for 15 min. The reaction mixture was concentrated and used directly in the next step as the sodium salt without additional purification (quantitative yield assumed). LC/MS (ESI) m/z: [M+H]⁺ calcd for C₆H₅FNO₃ 158.1; found 157.8.

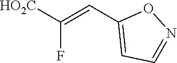 [SMILES: O=C(O)/C(F)=C/c1ccno1]

### Intermediate I

### (Z)-2-fluoro-3-(isoxazol-5-yl)acrylic acid

### Step 1: Preparation of ethyl (Z)-2-fluoro-3-(isoxazol-5-yl)acrylate

(2-ethoxy-1-fluoro-2-oxoethyl)triphenylphosphonium bromide (922 mg, 2.06 mmol) was stirred in THE (10 mL) at 0° C. To the mixture was added dropwise nBuLi solution (2.5 M in hexanes, (0.82 mL, 2.06 mmol). The mixture was stirred for 10 minutes and then isoxazole-5-carbaldehyde (100 mg, 1.030 mmol) was added as a solid. The reaction mixture was allowed to warm to room temperature over two hours. The reaction mixture was concentrated, and the residue was directly purified by column chromatography (5→100% EtOAc/hexanes) to afford ethyl (Z)-2-fluoro-3-(isoxazol-5-yl)acrylate (31 mg, 0.11 mmol, 16% yield). LC/MS (ESI) m/z: [M+H]⁺ calcd for C₈H₉FNO₃ 186.1; found 185.9; ¹H NMR (500 MHz, CDCl₃) δ 8.32 (dd, J=1.7, 0.8 Hz, 1H), 7.09 (d, J=31.6 Hz, 1H), 6.71 (t, J=1.7 Hz, 1H), 4.38 (q, J=7.2 Hz, 2H), 1.39 (t, J=7.2 Hz, 3H).

### Step 2: Preparation of (Z)-2-fluoro-3-(isoxazol-5-yl)acrylic acid

Ethyl (Z)-2-fluoro-3-(isoxazol-5-yl)acrylate (31 mg, 0.11 mmol) was dissolved in MeOH (1.7 mL) and sodium hydroxide solution (1.0 M, 230 μL, 0.23 mmol) was added dropwise. The mixture was stirred for 15 min. The reaction mixture was concentrated and used directly in the next step as the sodium salt without additional purification (quantitative yield assumed). LC/MS (ESI) m/z: [M+H]⁺ calcd for C₆H₅FNO₃ 158.1; found 157.9.

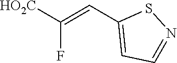 [SMILES: O=C(O)/C(F)=C/c1ccns1]

### Intermediate J

### (Z)-2-fluoro-3-(isothiazol-5-yl)acrylic acid

### Step 1: Preparation of ethyl (Z)-2-fluoro-3-(isothiazol-5-yl)acrylate

(2-ethoxy-1-fluoro-2-oxoethyl)triphenylphosphonium bromide (790 mg, 1.77 mmol) was stirred in THE (9 mL) at 0° C. To the mixture was added dropwise nBuLi solution (2.5 M in hexanes, (0.71 mL, 1.77 mmol). The mixture was stirred for 10 minutes and then isothiazol-5-carbaldehyde (100 mg, 0.88 mmol) was added as a solution in THE (1 mL). The reaction mixture was allowed to warm to room temperature over two hours. The reaction mixture was concentrated, and the residue was directly purified by column chromatography (5→100% EtOAc/hexanes) to afford ethyl (Z)-2-fluoro-3-(isothiazol-5-yl)acrylate (71 mg, 0.35 mmol, 40% yield). LC/MS (ESI) m/z: [M+H]⁺ calcd for C₈H₉FNO₂S 202.0; found 201.9; ¹H NMR (500 MHz, CDCl₃) δ 8.49 (t, J=2.0 Hz, 1H), 7.39 (dd, J=2.0, 1.0 Hz, 1H), 7.27 (d, J=33.4 Hz, 1H), 4.38 (q, J=7.2 Hz, 2H), 1.39 (t, J=7.2 Hz, 3H).

### Step 2: Preparation of (Z)-2-fluoro-3-(isothiazol-5-yl)acrylic acid

Ethyl (Z)-2-fluoro-3-(isothiazol-5-yl)acrylate (71 mg, 0.35 mmol) was dissolved in MeOH (3.5 mL) and sodium hydroxide solution (1.0 M, 420 μL, 0.420 mmol) was added dropwise. The mixture was stirred for 2 h. The reaction mixture was concentrated and used directly in the next step as the sodium salt without additional purification (quantitative yield assumed). LC/MS (ESI) m/z: [M+H]⁺ calcd for C₆H₅FNO₂S 174.1; found 173.9.

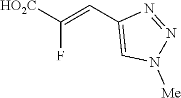 [SMILES: Cn1cc(/C=C(\F)C(=O)O)nn1]

### Intermediate K

### (Z)-2-fluoro-3-(1-methyl-1H-1,2,3-triazol-4-yl)acrylic acid

### Step 1: Preparation of ethyl (Z)-2-fluoro-3-(1-methyl-1H-1,2,3-triazol-4-yl)acrylate

(2-ethoxy-1-fluoro-2-oxoethyl)triphenylphosphonium bromide (1.1 g, 2.5 mmol) was stirred in THE (10 mL) at 0° C. To the mixture was added dropwise nBuLi solution (2.5 M in hexanes, (1.0 mL, 2.5 mmol). The mixture was stirred for 10 minutes and then 1-methyl-1H-1,2,3-triazol-4-yl-4-carbaldehyde (140 mg, 1.26 mmol) was added as a solution in THE (2 mL). The reaction mixture was allowed to warm to room temperature over two hours. The reaction mixture was concentrated, and the residue was directly purified by column chromatography (5→100% EtOAc/hexanes) to afford ethyl (Z)-2-fluoro-3-(1-methyl-1H-1,2,3-triazol-4-yl)acrylate (132 mg, 0.66 mmol, 53% yield). LC/MS (ESI) m/z: [M+H]⁺ calcd for C₈H₁₁FN₃O₂ 200.1; found 199.9; ¹H NMR (500 MHz, CDCl₃) δ 7.93 (d, J=1.9 Hz, 1H), 7.24 (d, J=34.9 Hz, 1H), 4.33 (q, J=7.2 Hz, 2H), 4.14 (s, 3H), 1.35 (t, J=7.2 Hz, 3H).

### Step 2: Preparation of (Z)-2-fluoro-3-(1-methyl-1H-1,2,3-triazol-4-yl)acrylic acid

Ethyl (Z)-2-fluoro-3-(1-methyl-1H-1,2,3-triazol-4-yl)acrylate (132 mg, 0.66 mmol) was dissolved in MeOH (5 mL) and sodium hydroxide solution (1.0 M, 920 μL, 0.92 mmol) was added dropwise. The mixture was stirred for 2 h. The reaction mixture was concentrated, diluted with water (1 mL), and hydrochloric acid solution (1.0 M, 920 μL, 0.92 mmol) was added. This solution was purified by HPLC (column: Xbridge C18, 19 mm×200 mm, 5 m particles; flow rate: 42.5 mL/min; column temperature: 25° C.; gradient: 5:95 MeCN:H₂O with 10 mM AA→95:5 MeCN:H₂O with 10 mM AA; λ=220, 254 nm) to afford (Z)-2-fluoro-3-(1-methyl-1H-1,2,3-triazol-4-yl)acrylic acid (11 mg, 0.06 mmol, 10% yield). LC/MS (ESI) m/z: [M+H]⁺ calcd for C₆H₇FN₃O₂ 172.0; found 171.9; ¹H NMR (500 MHz, CD₃OD) δ 8.28 (s, 1H), 7.09 (d, J=34.0 Hz, 1H), 4.15 (s, 3H).

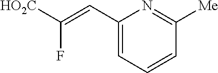 [SMILES: Cc1cccc(/C=C(\F)C(=O)O)n1]

### Intermediate L

### (Z)-2-fluoro-3-(6-methylpyridin-2-yl)acrylic acid

### Step 1: Preparation of ethyl (Z)-2-fluoro-3-(6-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 6-methylpyridine-2-carbaldehyde (125 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by HPLC (column: Xbridge C18, 5 m particles; gradient: 5:95 MeCN:H₂O with 0.05% TFA→95:5 MeCN:H₂O with 0.05% TFA; λ=220 nm) to afford ethyl (Z)-2-fluoro-3-(6-methylpyridin-2-yl)acrylate (31 mg, 0.15 mmol, 14% yield). LC/MS (ESI) m/z: [M+H]⁺ calcd for C₁₁H₁₃FNO₂ 210.1; found 209.7; ¹H NMR (500 MHz, CDCl₃) δ 7.71 (d, J=7.8 Hz, 1H), 7.63 (t, J=7.8 Hz, 1H), 7.10 (d, J=7.8 Hz, 1H), 7.11 (d, J=35.2 Hz, 1H), 4.34 (q, J=7.2 Hz, 2H), 2.56 (s, 3H), 1.36 (t, J=7.2 Hz, 3H).

### Step 2: Preparation of (Z)-2-fluoro-3-(6-methylpyridin-2-yl)acrylic acid

Ethyl (Z)-2-fluoro-3-(6-methylpyridin-4-yl)acrylate (31 mg, 0.15 mmol) was dissolved in MeOH (1.5 mL) and sodium hydroxide solution (1.0 M, 150 μL, 0.15 mmol) was added dropwise. The mixture was stirred for 2 h. The reaction mixture was concentrated, diluted with water (1 mL), and hydrochloric acid solution (1.0 M, 150 μL, 0.14 mmol) was added. This solution was lyophilized and the crude material was used without additional purification (quantitative yield assumed). LC/MS (ESI) m/z: [M+H]⁺ calcd for C₉H₉FNO₂ 182.1; found 182.1; ¹H NMR (500 MHz, DMSO-d₆) δ 7.80 (t, J=7.7 Hz, 1H), 7.65 (d, J=7.7 Hz, 1H), 7.29 (d, J=7.7 Hz, 1H), 6.94 (d, J=35.4 Hz, 1H), 2.50 (s, 3H).

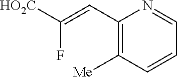 [SMILES: Cc1cccnc1/C=C(\F)C(=O)O]

### Intermediate M

### (Z)-2-fluoro-3-(3-methylpyridin-2-yl)acrylic acid

### Step 1: Preparation of ethyl (Z)-2-fluoro-3-(3-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 3-methylpyridine-2-carbaldehyde (125 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by HPLC (column: Xbridge C18, 5 m particles; gradient: 5:95 MeCN:H₂O with 0.05% TFA→95:5 MeCN:H₂O with 0.05% TFA; λ=220 nm) to afford ethyl (Z)-2-fluoro-3-(3-methylpyridin-2-yl)acrylate (30 mg, 0.14 mmol, 13% yield). LC/MS (ESI) m/z: [M+H]⁺ calcd for C₁₁H₁₃FNO₂ 210.1; found 209.7; ¹H NMR (500 MHz, CDCl₃) δ 8.53 (dd, J=4.2, 0.8 Hz, 1H), 7.52 (dd, J=7.7, 0.8 Hz, 1H), 7.16 (dd, J=7.7, 4.2 Hz, 1H), 7.15 (d, J=32.4 Hz, 1H), 4.37 (q, J=7.2 Hz, 2H), 2.37 (s, 3H), 1.38 (t, J=7.2 Hz, 3H).

### Step 2: Preparation of (Z)-2-fluoro-3-(3-methylpyridin-2-yl)acrylic acid

Ethyl (Z)-2-fluoro-3-(3-methylpyridin-4-yl)acrylate (30 mg, 0.14 mmol) was dissolved in MeOH (1.5 mL) and sodium hydroxide solution (1.0 M, 140 μL, 0.15 mmol) was added dropwise. The mixture was stirred for 2 h. The reaction mixture was concentrated, diluted with water (1 mL), and hydrochloric acid solution (1.0 M, 140 μL, 0.14 mmol) was added. This solution was lyophilized and the crude material was used without additional purification (quantitative yield assumed). LC/MS (ESI) m/z: [M+H]⁺ calcd for C₉H₉FNO₂ 182.1; found 181.8; NMR: ¹H NMR (500 MHz, DMSO-d₆) δ 8.48 (dd, J=4.3, 0.8 Hz, 1H), 7.68 (dd, J=7.7, 0.8 Hz, 1H), 7.29 (dd, J=7.7, 4.3 Hz, 1H), 7.11 (d, J=32.9 Hz, 1H), 2.33 (s, 3H).

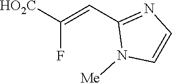 [SMILES: Cn1ccnc1/C=C(\F)C(=O)O]

### Intermediate N

### (Z)-2-fluoro-3-(1-methyl-1H-imidazol-2-yl)acrylic acid

### Step 1: Preparation of ethyl (Z)-2-fluoro-3-(1-methyl-1H-imidazol-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 1-methylimidazole-2-carbaldehyde (114 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by reverse phase HPLC (column: Xbridge C18, 30 mm×100 mm, 5 m particles; flow rate: 42.5 mL/min; column temperature: 25° C.; gradient: 95:5 H₂O:MeCN:10 mM AA→100% 5:95 H₂O:MeCN:10 nM AA; λ=220 nm) to afford ethyl (Z)-2-fluoro-3-(1-methyl-1H-imidazol-2-yl)acrylate (16 mg, 0.08 mmol, 8% yield). LC/MS (ESI) m/z: [M+H]⁺ calcd for C₉H₁₂FN₂O₂ 199.1; found 198.9; ¹H NMR (500 MHz, CDCl₃) δ 7.22 (d, J=0.7 Hz, 1H), 6.94 (d, J=0.7 Hz, 1H), 6.84 (d, J=30.5 Hz, 1H), 4.33 (q, J=7.1 Hz, 2H), 3.71 (s, 3H), 1.35 (t, J=7.1 Hz, 3H).

### Step 2: Preparation of (Z)-2-fluoro-3-(1-methyl-1H-imidazol-2-yl)acrylic acid

Ethyl (Z)-2-fluoro-3-(1-methyl-1H-imidazol-2-yl)acrylate (16 mg, 0.08 mmol) was dissolved in MeOH (1 mL). The solution was cooled to 0° C. and sodium hydroxide solution (1.0 M, 0.08 mL, 0.08 mmol) was added. The reaction mixture was stirred for 2 h. The solution was concentrated to remove the methanol. Additional water (0.3 mL) was added and the aqueous solution was cooled to 0° C. HCl solution (1.0 M, 0.08 mL, 0.08 mmol) was added dropwise. The solution was frozen and directly lyophilized to afford (Z)-2-fluoro-3-(1-methyl-1H-imidazol-2-yl)acrylic acid (13 mg, 0.08 mmol, quantitative yield assumed; contains 1 equiv NaCl) as a white solid. LC/MS (ESI) m/z: [M+H]⁺ calcd for C₇H₈FN₂O₂ 171.0; found 170.9; ¹H NMR (500 MHz, DMSO-d₆) δ 7.31 (s, 1H), 7.11 (s, 1H), 6.87 (d, J=31.6 Hz, 1H), 3.72 (s, 3H).

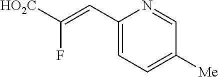 [SMILES: Cc1ccc(/C=C(\F)C(=O)O)nc1]

### Intermediate U

### (Z)-2-fluoro-3-(5-methylpyridin-2-yl)acrylic acid

### Step 1: Preparation of ethyl (Z)-2-fluoro-3-(5-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 5-methylpicolinaldehyde (125 mg, 1.0 mmol) was added. The reaction mixture was allowed to warm to room temperature and was stirred for 1 h. The mixture was quenched by addition of saturated aqueous ammonium chloride solution (15 mL). The solution was diluted with water (20 mL) and EtOAc (100 mL). The layers were separated and the aqueous phase was further extracted with EtOAc (2×50 mL). The combined organic layers were dried over magnesium sulfate, filtered, and concentrated. The crude residue was purified by reverse phase HPLC (column: Xbridge C18, 30 mm×100 mm, 5 m particles; flow rate: 42.5 mL/min; column temperature: 25° C.; gradient: 95:5 H₂O:MeCN:0.05% TFA→100% 5:95 H₂O:MeCN:0.05% TFA; λ=220 nm) to afford ethyl (Z)-2-fluoro-3-(4-methylpyridin-2-yl)acrylate (12 mg, 0.06 mmol, 6% yield). LC/MS (ESI) m/z: [M+H]⁺ calcd for C₁₁H₁₃FNO₂ 210.2; found 210.1 ¹H NMR (500 MHz, CDCL₃) δ 8.49 (d, J=1.8 Hz, 1H), 7.79 (d, J=8.2 Hz, 1H), 7.55 (dd, J=8.2, 1.8 Hz, 1H), 7.12 (d, J=35.3 Hz, 1H), 4.35 (q, J=7.2 Hz, 2H), 2.37 (s, 3H), 1.37 (t, J=7.2 Hz, 3H).

### Step 2: Preparation of (Z)-2-fluoro-3-(5-methylpyridin-2-yl)acrylic acid

Ethyl (Z)-2-fluoro-3-(5-methylpyridin-2-yl)acrylate (12 mg, 0.06 mmol) was dissolved in MeOH (0.5 mL). The solution was cooled to 0° C. and sodium hydroxide solution (1.0 M, 0.06 mL, 0.06 mmol) was added. The reaction mixture was stirred for 2 h. The solution was concentrated to remove the methanol. Additional water (1.0 mL) was added and the aqueous solution was cooled to 0° C. HCl solution (1.0 M, 0.06 mL, 0.06 mmol) was added dropwise. The solution was frozen and directly lyophilized to afford (Z)-2-fluoro-3-(5-methylpyridin-2-yl)acrylic acid (10 mg, 0.06 mmol, quantitative yield assumed; contains 1 equiv NaCl) as a white solid. LC/MS (ESI) m/z: [M+H]⁺ calcd for C₉H₈FNO₂ 182.1; found 181.6; ¹H NMR (500 MHz, DMSO-d₆) δ 8.51 (s, 1H), 7.74-7.69 (m, 2H), 6.95 (d, J=35.4 Hz, 1H), 2.33 (s, 3H).

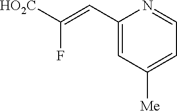 [SMILES: Cc1ccnc(/C=C(\F)C(=O)O)c1]

### Intermediate P

### (Z)-2-fluoro-3-(4-methylpyridin-2-yl)acrylic acid

### Step 1: Preparation of ethyl (Z)-2-fluoro-3-(4-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 2-formyl-4-picoline (125 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by reverse phase HPLC (column: Xbridge C18, 30 mm×100 mm, 5 m particles; flow rate: 42.5 mL/min; column temperature: 25° C.; gradient: 95:5 H₂O:MeCN:0.05% TFA→5:95 H₂O:MeCN:0.05% TFA; λ=220 nm) to afford ethyl (Z)-2-fluoro-3-(4-methylpyridin-2-yl)acrylate (32 mg, 0.15 mmol, 15% yield). LC/MS (ESI) m/z: [M+H]⁺ calcd for C₁₁H₁₃FNO₂ 210.2; found 209.8; ¹H NMR (500 MHz, CDCl₃) δ 8.51 (d, J=5.0 Hz, 1H), 7.70 (s, 1H), 7.11 (d, J=35.0 Hz, 1H), 7.07 (d, J=5.0 Hz, 2H), 4.35 (q, J=7.2 Hz, 2H), 2.40 (s, 3H), 1.37 (t, J=7.2 Hz, 3H).

### Step 2: Preparation of (Z)-2-fluoro-3-(4-methylpyridin-2-yl)acrylic acid

Ethyl (Z)-2-fluoro-3-(4-methylpyridin-2-yl)acrylate (32 mg, 0.15 mmol) was dissolved in MeOH (1.5 mL). The solution was cooled to 0° C. and sodium hydroxide solution (1.0 M, 0.15 mL, 0.15 mmol) was added. The reaction mixture was stirred for 2 h. The solution was concentrated to remove the methanol. Additional water (1.0 mL) was added and the aqueous solution was cooled to 0° C. HCl solution (1.0 M, 0.15 mL, 0.15 mmol) was added dropwise. The solution was frozen and directly lyophilized to afford (Z)-2-fluoro-3-(4-methylpyridin-2-yl)acrylic acid (27 mg, 0.15 mmol, quantitative yield assumed; contains 1 equiv NaCl) as a white solid. LC/MS (ESI) m/z: [M+H]⁺ calcd for C₉H₈FNO₂ 182.1; found 182.1. ¹H NMR (500 MHz, DMSO-d₆) δ 8.51 (d, J=4.9 Hz, 1H), 7.65 (s, 1H), 7.24 (d, J=4.9 Hz, 1H), 6.94 (d, J=35.3 Hz, 1H), 2.36 (s, 3H).

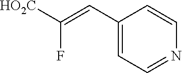 [SMILES: O=C(O)/C(F)=C/c1ccncc1]

### Intermediate Q

### (Z)-2-fluoro-3-(pyridin-4-yl)acrylic acid

### Step 1: Preparation of ethyl (Z)-2-fluoro-3-(pyridin-4-yl)acrylate

Isonicotinaldehyde (100 mg, 0.9 mmol) and ethyl 2-fluoroacetate (200 mg, 1.9 mmol) were dissolved in THE (4.7 mL). Ethyl 2-fluoroacetate (198 mg, 1.867 mmol) was added and the reaction mixture was heated at 60° C. for 1 h. The reaction mixture was quenched by addition of a saturated aqueous solution of ammonium chloride (5 mL). Ethyl acetate (5 mL) was added and the layers were separated. The organic phase was dried over sodium sulfate, filtered, and concentrated. The crude residue was purified by column chromatography (30→50% EtOAc/hexanes) to afford ethyl (Z)-2-fluoro-3-(pyridin-4-yl)acrylate (62 mg, 0.318 mmol, 34.0% yield). LC/MS (ESI) m/z: [M+H]⁺ calcd for C₁₀H₁₁FNO₂ 196.1; found 195.6. ¹H NMR (500 MHz, CDCl₃) δ 8.68-8.65 (m, 2H), 7.48-7.45 (m, 2H), 6.85 (d, J=33.7 Hz, 1H), 4.37 (q, J=7.2 Hz, 2H), 1.37 (t, J=7.2 Hz, 3H).

### Step 2: Preparation of (Z)-2-fluoro-3-(pyridin-4-yl)acrylic acid

Ethyl (Z)-2-fluoro-3-(pyridin-4-yl)acrylate (60 mg, 0.3 mmol) was dissolved in MeOH (1.5 mL) and an aqueous solution of sodium hydroxide (1.0 M, 0.46 mL, 0.46 mmol) was added dropwise. The reaction mixture was stirred for 1 h and was concentrated. An aqueous solution of hydrochloric acid (1.0 M, 0.46 mL, 0.46 mmol) was added dropwise. The solution was frozen and lyophilized to afford (Z)-2-fluoro-3-(pyridin-4-yl)acrylic acid as a white solid (quantitative yield assumed). LC/MS (ESI) m/z: [M+H]⁺ calcd for C₈H₇FNO₂ 168.0; found 168.0.

### Preparation of KRAS Inhibitors

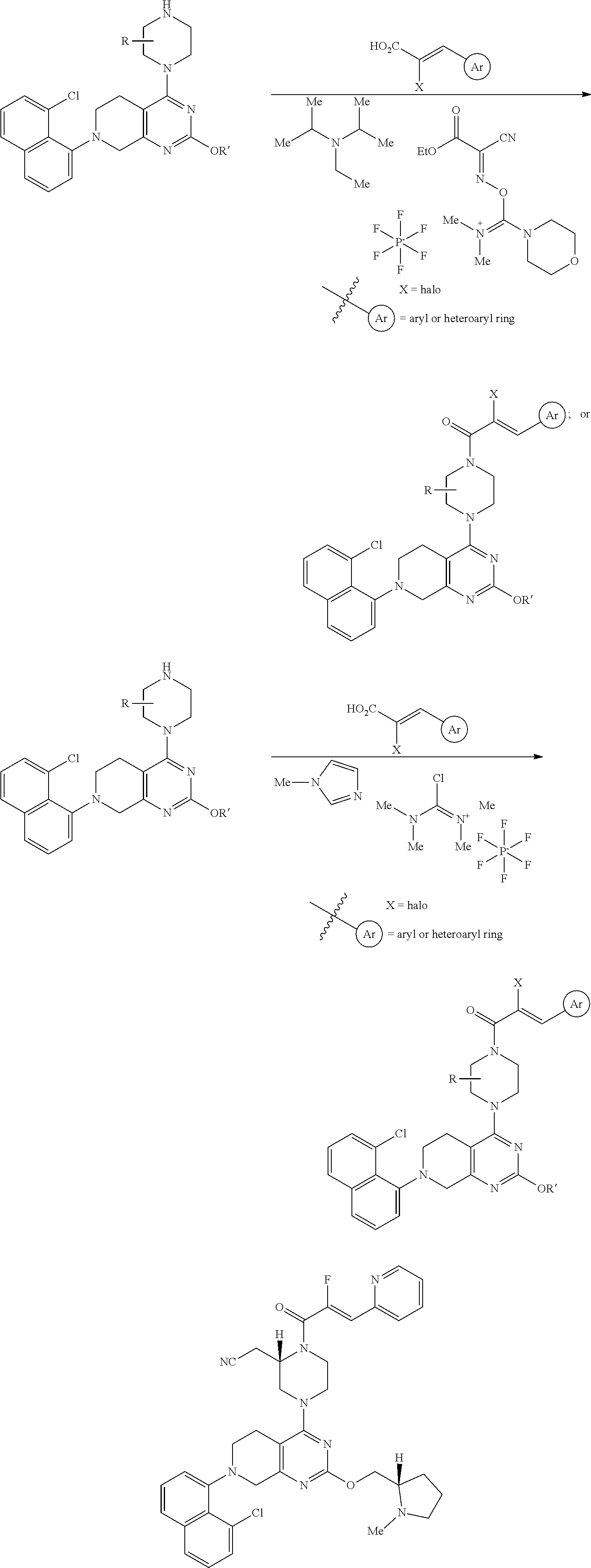

### Example 1

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile

2-((S)-4-(7-(8-Chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Intermediate A, 15 mg, 0.028 mmol) and (Z)-2-fluoro-3-(pyridin-2-yl)acrylic acid (Intermediate C, 14 mg, 0.085 mmol) were combined as solids and dissolved in DMF (1 mL). DIPEA (0.020 mL, 0.113 mmol) was added followed by COMU® ((1-cyano-2-ethoxy-2-oxoethylidenaminooxy)dimethylaminomorpholino-carbenium hexafluorophosphate) (36.2 mg, 0.085 mmol), and the reaction mixture was stirred at room temperature overnight. The solution was concentrated directly and purified by HPLC (column: Xbridge C18, 5 m particles; gradient: 5:95 MeCN:H₂O with 0.05% TFA→95:5 MeCN:H₂O with 0.05% TFA; λ=220 nm) to provide 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile, 2 TFA salt (4.1 mg, 4.19 μmol, 15% yield) as an off-white solid. LC/MS (ESI) m/z: [M+H]⁺ cacld for C₃₇H₃₉ClFN₈O₂ 681.3; found 681.1; ¹H NMR (500 MHz, DMSO-d₆) δ 8.65 (d, J=4.1 Hz, 1H), 7.93-7.86 (m, 2H), 7.81-7.77 (m, 1H), 7.76-7.71 (m, 1H), 7.58 (dt, J=7.5, 1.3 Hz, 1H), 7.56-7.51 (m, 1H), 7.47-7.42 (m, 1H), 7.40-7.31 (m, 2H), 6.61 (d, J=38.9 Hz, 1H), 5.03-4.58 (m, 1H), 4.28-4.13 (m, 2H), 4.10-3.88 (m, 4H), 3.83-3.69 (m, 2H), 3.56-3.46 (m, 2H), 3.31 (br d, J=3.7 Hz, 1H), 3.19-3.04 (m, 4H), 3.01-2.88 (m, 2H), 2.75-2.65 (m, 1H), 2.56-2.51 (m, 1H), 2.32 (s, 3H), 2.15 (qd, J=8.6, 2.6 Hz, 1H), 1.95-1.90 (m, 1H), 1.70-1.62 (m, 2H), 1.61-1.53 (m, 1H).

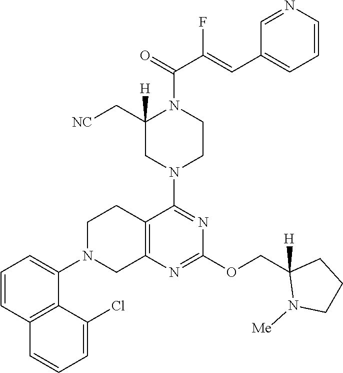 [SMILES: CN1CCC[C@H]1COc1nc2c(c(N3CCN(C(=O)/C(F)=C/c4cccnc4)[C@@H](CC#N)C3)n1)CCN(c1cccc3cccc(Cl)c13)C2]

### Example 2

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-3-yl)acryloyl)piperazin-2-yl)acetonitrile

Prepared by a procedure analogous to the one used for Example 1. LC/MS (ESI) m/z: [M+H]⁺ cacld for C₃₇H₃₉ClFN₈O₂ 681.3; found 681.2.

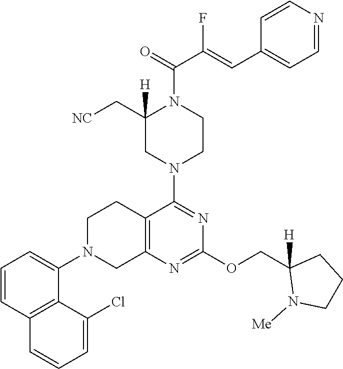 [SMILES: CN1CCC[C@H]1COc1nc2c(c(N3CCN(C(=O)/C(F)=C/c4ccncc4)[C@@H](CC#N)C3)n1)CCN(c1cccc3cccc(Cl)c13)C2]

### Example 3

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-4-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Intermediate A, 10 mg, 0.019 mmol) and (Z)-2-fluoro-3-(pyridin-4-yl)acrylic acid (6 mg, 0.038 mmol) were combined as solids and dissolved in DMF (300 L). DIPEA (10 μL, 0.056 mmol) was added followed by COMU® ((1-cyano-2-ethoxy-2-oxoethylidenaminooxy)dimethylaminomorpholinocarbenium hexafluorophosphate) (12 mg, 0.028 mmol), and the reaction mixture was stirred at room temperature for 10 min. The reaction mixture was directly purified by reverse phase HPLC (column: Xbridge C18, 19 mm×200 mm, 5 m particles; flow rate: 20 mL/min; column temperature: 25° C.; gradient: 88% 5:95 MeCN:H₂O with 0.1% TFA/12% 95:5 MeCN:H₂O with 0.1% TFA→100% 95:5 MeCN:H₂O with 0.1% TFA; λ=220 nm) to provide 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-4-yl)acryloyl)piperazin-2-yl)acetonitrile, TFA salt (10.6 mg, 0.013 mmol, 70% yield) as a white solid. LC/MS (ESI) m/z: [M+H]⁺ cacld for C₃₇H₃₉ClFN₈O₂ 681.3; found 681.2; NMR: N.B. Spectrum reported as obtained; a water suppression method was used; presence of atropisomers and amide rotamers complicate analysis. ¹H NMR (500 MHz, DMSO-d₆) δ 8.76 (br s, 2H), 7.93 (br d, J=8.1 Hz, 1H), 7.78 (br s, 2H), 7.77-7.73 (m, 1H), 7.61-7.57 (m, 1H), 7.56-7.51 (m, 1H), 7.49-7.42 (m, 1H), 7.39-7.32 (m, 1H), 6.79 (d, J=38.1 Hz, 1H), 4.98-4.89 (m, 1H), 4.61-4.54 (m, 1H), 4.48-4.39 (m, 1H), 4.26-4.13 (m, 3H), 4.11-3.93 (m, 2H), 3.83-3.73 (m, 2H), 3.25-3.06 (m, 1H), 3.00-2.95 (m, 1H), 2.93 (br s, 3H), 2.80-2.69 (m, 1H), 2.62-2.52 (m, 1H), 2.29-2.20 (m, 1H), 2.09-1.99 (m, 1H), 1.96-1.75 (m, 3H).

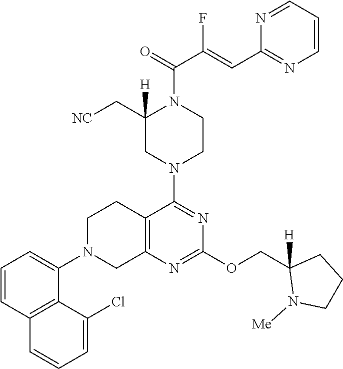 [SMILES: CN1CCC[C@H]1COc1nc2c(c(N3CCN(C(=O)/C(F)=C/c4ncccn4)[C@@H](CC#N)C3)n1)CCN(c1cccc3cccc(Cl)c13)C2]

### Example 4

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyrimidin-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Intermediate A, 10 mg, 0.019 mmol) and (Z)-2-fluoro-3-(pyrimidin-2-yl)acrylic acid (Intermediate D, 6 mg, 0.038 mmol) were combined as solids and dissolved in DMF (300 L). DIPEA (10 μL, 0.056 mmol) was added followed by COMU® ((1-cyano-2-ethoxy-2-oxoethylidenaminooxy)dimethylaminomorpholino-carbenium hexafluorophosphate) (12 mg, 0.028 mmol), and the reaction mixture was stirred at room temperature for 10 min. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 0.1% TFA/21% 95:5 MeCN:H₂O with 0.1% TFA→100% 95:5 MeCN:H₂O with 0.1% TFA; λ=220 nm) to provide 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyrimidin-2-yl)acryloyl)piperazin-2-yl)acetonitrile, TFA salt (4.5 mg, 0.006 mmol, 30% yield) as a white solid. LC/MS (ESI) m/z: [M+H]⁺ cacld for C₃₆H₃₈ClFN₉O₂ 682.3; found 682.2 NMR: N.B. Spectrum reported as obtained; a water suppression method was used; presence of atropisomers and amide rotamers complicate analysis. ¹H NMR (500 MHz, DMSO-d₆) δ 8.89 (d, J=4.8 Hz, 2H), 7.93 (d, J=8.1 Hz, 1H), 7.78-7.73 (m, 1H), 7.60-7.56 (m, 1H), 7.56-7.52 (m, 1H), 7.46 (t, J=4.8 Hz, 1H), 7.49-7.42 (m, 1H), 7.39-7.31 (m, 1H), 6.58 (br d, J=35.2 Hz, 1H), 4.57 (br d, J=12.2 Hz, 1H), 4.49-4.40 (m, 1H), 4.24-3.96 (m, 3H), 3.83-3.70 (m, 2H), 3.67-3.53 (m, 1H), 3.24-3.05 (m, 5H), 3.00-2.95 (m, 1H), 2.93 (br s, 3H), 2.81-2.70 (m, 1H), 2.28-2.19 (m, 1H), 2.08-2.00 (m, 1H), 1.94-1.78 (m, 2H).

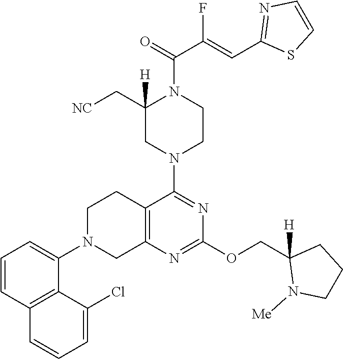 [SMILES: CN1CCC[C@H]1COc1nc2c(c(N3CCN(C(=O)/C(F)=C/c4nccs4)[C@@H](CC#N)C3)n1)CCN(c1cccc3cccc(Cl)c13)C2]

### Example 5

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (20 mg, 0.019 mmol) and (Z)-2-fluoro-3-(thiazol-2-yl)acrylic acid (Intermediate E, 6 mg, 0.038 mmol) were combined as solids and dissolved in DMF (300 L). DIPEA (20 μL, 0.056 mmol) was added followed by COMU® ((1-cyano-2-ethoxy-2-oxoethylidenaminooxy)dimethylamino-morpholino-carbenium hexafluorophosphate) (16 mg, 0.028 mmol), and the reaction mixture was stirred at room temperature for 15 min. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile (5.6 mg, 0.008 mmol, 22% yield) as a white solid. LC/MS (ESI) m/z: [M+H]⁺ cacld for C₃₅H₃₇ClFN₈O₂S 687.2; found 687.2; NMR: N.B. Spectrum reported as obtained; a water suppression method was used; presence of atropisomers and amide rotamers complicate analysis. ¹H NMR (500 MHz, DMSO-d₆) δ 8.04-7.99 (m, 2H), 7.92 (d, J=8.2 Hz, 1H), 7.77-7.72 (m, 1H), 7.61-7.51 (m, 2H), 7.47-7.42 (m, 1H), 7.38-7.31 (m, 1H), 7.06 (d, J=38.8 Hz, 1H), 5.02-4.30 (m, 1H), 4.29-3.89 (m, 6H), 3.83-3.70 (m, 1H), 3.53-3.44 (m, 1H), 3.19-2.88 (m, 3H), 2.75-2.61 (m, 1H), 2.36 (br s, 3H), 2.28-2.12 (m, 1H), 1.96-1.91 (m, 1H), 1.72-1.54 (m, 3H).

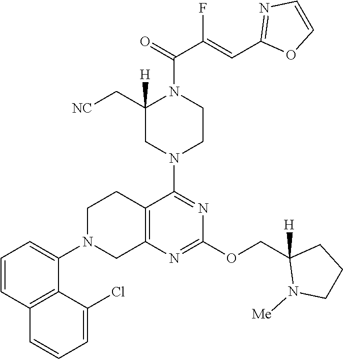 [SMILES: CN1CCC[C@H]1COc1nc2c(c(N3CCN(C(=O)/C(F)=C/c4ncco4)[C@@H](CC#N)C3)n1)CCN(c1cccc3cccc(Cl)c13)C2]

### Example 6

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(oxazol-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (Intermediate A, 10 mg, 0.019 mmol) and (Z)-2-fluoro-3-(oxazol-2-yl)acrylic acid (Intermediate F, 4 mg, 0.038 mmol) were combined as solids and dissolved in DMF (0.5 mL). 1-methylimidazole (0.015 mL, 0.188 mmol) was added followed by chloro-N,N,N′,N′-tetramethylformamidinium hexafluorophospate (5.3 mg, 0.019 mmol) and the reaction mixture was stirred at room temperature for 1 h. The reaction mixture was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(oxazol-2-yl)acryloyl)piperazin-2-yl)acetonitrile (2.9 mg, 0.004 mmol, 23% yield) as a white solid. LC/MS (ESI) m/z: [M+H]⁺ cacld for C₃₅H₃₇ClFN₈O₃ 671.3; found 671.2; NMR: N.B. Spectrum reported as obtained; a water suppression method was used; presence of atropisomers and amide rotamers complicate analysis. ¹H NMR (500 MHz, DMSO-d₆) δ 8.28 (s, 1H), 7.92 (d, J=8.2 Hz, 1H), 7.78-7.71 (m, 1H), 7.63-7.48 (m, 2H), 7.45 (s, 1H), 7.48-7.42 (m, 1H), 7.42-7.28 (m, 1H), 6.67 (br d, J=35.1 Hz, 1H), 5.00-4.37 (m, 1H), 4.28-4.11 (m, 2H), 4.09-3.92 (m, 3H), 3.85-3.70 (m, 1H), 3.53-3.43 (m, 1H), 3.15-3.04 (m, 2H), 3.00-2.88 (m, 1H), 2.72-2.55 (m, 1H), 2.44-2.35 (m, 1H), 2.33 (br s, 3H), 2.21-2.11 (m, 1H), 1.96-1.90 (m, 2H), 1.71-1.63 (m, 2H), 1.60-1.52 (m, 1H).

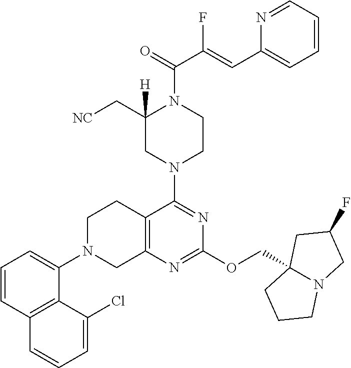 [SMILES: N#CC[C@H]1CN(c2nc(OC[C@@]34CCCN3C[C@H](F)C4)nc3c2CCN(c2cccc4cccc(Cl)c24)C3)CCN1C(=O)/C(F)=C/c1ccccn1]

### Example 7

### 2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile

2-((S)-4-(7-(8-chloronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperazin-2-yl)acetonitrile (Intermediate B, 40 mg, 0.069 mmol) and (Z)-2-fluoro-3-(pyridin-2-yl)acrylic acid (Intermediate C, 5.8 mg, 0.035 mmol) were combined as solids and dissolved in DMF (3.5 mL). 1-methylimidazole (0.055 mL, 0.69 mmol) was added followed by chloro-N,N,N′,N′-tetramethylformamidinium hexafluorophospate (10 mg, 0.035 mmol) and the reaction mixture was stirred at room temperature for 5 min. Additional portions of (Z)-2-fluoro-3-(pyridin-2-yl)acrylic acid (5.8 mg, 0.035 mmol) and chloro-N,N,N′,N′-tetramethylformamidinium hexafluorophospate (10 mg, 0.035 mmol) were added and the reaction mixture was stirred for 5 min. This process was repeated an additional 6 times. The reaction mixture was directly purified by reverse phase HPLC (column: Xbridge C18, 19 mm×200 mm, 5 m particles; flow rate: 20 mL/min; column temperature: 25° C.; gradient: 65% 5:95 MeCN:H₂O with 10 mM AA/35% 95:5 MeCN:H₂O with 10 mM AA→100% 95:5 MeCN:H₂O with 10 mM AA; λ=220 nm) to provide 2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile (8.5 mg, 0.012 mmol, 17% yield) as a white solid. LC/MS (ESI) m/z: [M+H]⁺ cacld for C₃₉H₃₉ClF₂N₈O₂ 725.3; found 725.2; NMR: N.B. Spectrum reported as obtained; a water suppression method was used; presence of atropisomers and amide rotamers complicate analysis. ¹H NMR (500 MHz, DMSO-d₆) δ 8.65 (br d, J=4.1 Hz, 1H), 7.89 (t, J=7.6 Hz, 1H), 7.91 (d, J=8.1 Hz, 1H), 7.78 (d, J=7.7 Hz, 1H), 7.77-7.70 (m, 1H), 7.47-7.42 (m, 1H), 7.40-7.30 (m, 1H), 7.38 (dd, J=7.6, 4.1 Hz, 1H), 6.61 (d, J=39.2 Hz, 1H), 5.26 (d, J=53.7 Hz, 1H), 5.00-4.26 (m, 1H), 4.24-4.11 (m, 1H), 4.11-3.84 (m, 4H), 3.80-3.64 (m, 1H), 3.55-3.36 (m, 1H), 3.20-2.92 (m, 4H), 2.85-2.77 (m, 1H), 2.73-2.64 (m, 1H), 2.14-1.92 (m, 3H), 1.88-1.65 (m, 3H).

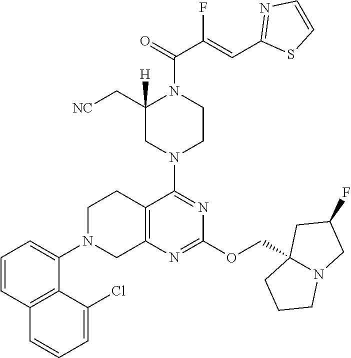 [SMILES: N#CC[C@H]1CN(c2nc(OC[C@@]34CCCN3C[C@H](F)C4)nc3c2CCN(c2cccc4cccc(Cl)c24)C3)CCN1C(=O)/C(F)=C/c1nccs1]

### Example 8

### 2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile

2-((S)-4-(7-(8-chloronaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperazin-2-yl)acetonitrile (Intermediate B, 7 mg, 0.012 mmol) and (Z)-2-fluoro-3-(thiazol-2-yl)acrylic acid (Intermediate E, 1.1 mg, 0.006 mmol) were combined as solids and dissolved in DMF (0.6 mL). 1-methylimidazole (0.01 mL, 0.122 mmol) was added followed by chloro-N,N,N′,N′-tetramethylformamidinium hexafluorophospate (1.7 mg, 0.006 mmol) and the reaction mixture was stirred at room temperature for 5 min. Additional portions of (Z)-2-fluoro-3-(thiazol-2-yl)acrylic acid (1.1 mg, 0.006 mmol) and chloro-N,N,N′,N′-tetramethylformamidinium hexafluorophospate (1.7 mg, 0.006 mmol) were added and the reaction mixture was stirred for 5 min. This process was repeated an additional 6 times. The reaction mixture was directly purified by reverse phase HPLC (column: Xbridge C18, 19 mm×200 mm, 5 μm particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide 2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile (1.4 mg, 0.002 mmol, 16% yield) as a white solid. LC/MS (ESI) m/z: [M+H]⁺ cacld for C₃₇H₃₈ClF₂N₈O₂S 731.2; found 731.2; NMR: N.B. Spectrum reported as obtained; a water suppression method was used; presence of atropisomers and amide rotamers complicate analysis. ¹H NMR (500 MHz, DMSO-d₆) δ 8.04-7.97 (m, 2H), 7.92 (d, J=8.2 Hz, 1H), 7.82-7.66 (m, 1H), 7.62-7.48 (m, 2H), 7.48-7.41 (m, 1H), 7.41-7.23 (m, 1H), 7.06 (br d, J=38.8 Hz, 1H), 5.26 (br d, J=54.1 Hz, 1H), 5.04-4.33 (m, 1H), 4.26-4.11 (m, 1H), 4.10-3.92 (m, 2H), 3.86-3.72 (m, 1H), 3.59-3.31 (m, 2H), 3.22-2.89 (m, 3H), 2.87-2.80 (m, 1H), 2.73-2.63 (m, 1H), 2.15-1.93 (m, 3H), 1.90-1.63 (m, 3H).

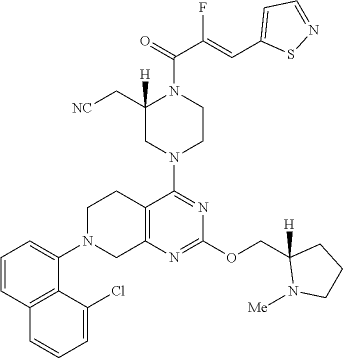 [SMILES: CN1CCC[C@H]1COc1nc2c(c(N3CCN(C(=O)/C(F)=C/c4ccns4)[C@@H](CC#N)C3)n1)CCN(c1cccc3cccc(Cl)c13)C2]

### Example 9

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoro-3-(isothiazol-5-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and 2-fluoro-3-(isothiazol-5-yl)acrylic acid (˜1.5:1 mixture of Z/E isomers, 7 mg, 0.038 mmol) were combined as solids and dissolved in DMF (1 mL). DIPEA (10 μL, 0.056 mmol) was added followed by COMU® ((1-Cyano-2-ethoxy-2-oxoethylidenaminooxy)dimethylamino-morpholino-carbenium hexafluorophosphate) (12 mg, 0.028 mmol), and the reaction mixture was stirred at room temperature overnight. The reaction mixture was directly purified by reverse phase HPLC (column: Xbridge C18, 19 mm×200 mm, 5 μm particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to afford the desired product (9.1 mg, 0.013 mmol, 68% yield, ˜1:1 mixture of Z/E isomers) as a white solid. LC/MS (ESI) m/z: [M+H]⁺ cacld for C₃₅H₃₇ClFN₈O₂S 687.2; found 687.2; NMR: N.B. Spectrum reported as obtained; two compounds are present, a (Z) alkene isomer and an (E) alkene isomer; a water suppression method was used; presence of atropisomers and amide rotamers complicate analysis. ¹H NMR (500 MHz, DMSO-d₆) δ 8.67-8.62 (m, 1H), 8.54-8.47 (m, 1H), 7.97-7.90 (m, 2H), 7.79-7.73 (m, 2H), 7.70-7.67 (m, 1H), 7.62-7.28 (m, 11H), 5.05-4.73 (m, 1H), 4.64-4.52 (m, 2H), 4.51-4.36 (m, 2H), 4.28-4.14 (m, 3H), 4.12-3.91 (m, 3H), 3.87-3.69 (m, 4H), 3.66-3.34 (m, 2H), 3.23-3.05 (m, 7H), 3.01-2.92 (m, 4H), 2.81-2.69 (m, 1H), 2.60-2.51 (m, 2H), 2.29-2.13 (m, 2H), 2.11-2.00 (m, 2H), 1.97-1.74 (m, 4H).

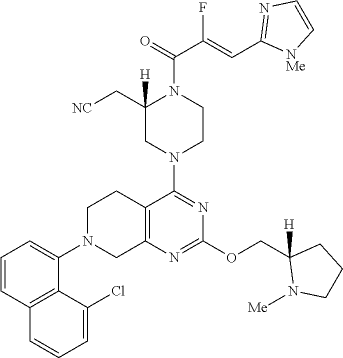 [SMILES: CN1CCC[C@H]1COc1nc2c(c(N3CCN(C(=O)/C(F)=C/c4nccn4C)[C@@H](CC#N)C3)n1)CCN(c1cccc3cccc(Cl)c13)C2]

### Example 10

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(1-methyl-1H-imidazol-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(1-methyl-1H-imidazol-2-yl)acrylic acid (4 mg, 0.019 mmol) were combined as solids and dissolved in DMF (0.5 mL). 1-methylimidazole (0.015 mL, 0.188 mmol) was added followed by chloro-N,N,N′,N′-tetramethylformamidinium hexafluorophospate (5 mg, 0.019 mmol) and the reaction mixture was stirred at room temperature for 1 h. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to afford the desired product (2.3 mg, 0.003 mmol, 17% yield) as a white solid. LC/MS (ESI) m/z: [M+H]⁺ cacld for C₃₆H₄₀ClFN₉O₂ 684.3; found 684.3; NMR: N.B. Spectrum reported as obtained; a water suppression method was used; presence of atropisomers and amide rotamers complicate analysis. ¹H NMR (500 MHz, DMSO-d₆) δ 7.92 (br d, J=8.0 Hz, 1H), 7.78-7.66 (m, 1H), 7.63-7.49 (m, 2H), 7.45 (t, J=7.8 Hz, 1H), 7.39-7.30 (m, 1H), 7.28 (s, 1H), 7.07 (s, 1H), 6.64 (d, J=35.0 Hz, 1H), 5.00-4.56 (m, 1H), 4.27-4.13 (m, 2H), 4.09-3.92 (m, 3H), 3.85-3.73 (m, 1H), 3.73-3.65 (m, 3H), 3.56-3.36 (m, 1H), 3.16-3.05 (m, 2H), 3.02-2.90 (m, 2H), 2.77-2.63 (m, 1H), 2.39-2.29 (m, 3H), 2.22-2.11 (m, 1H), 1.97-1.91 (m, 1H), 1.70-1.61 (m, 2H), 1.61-1.53 (m, 1H).

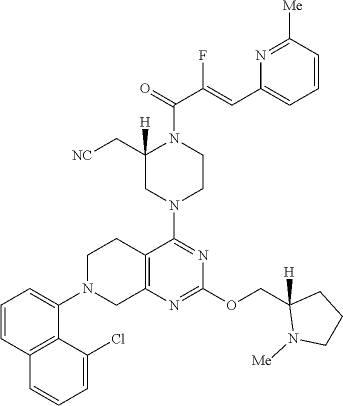 [SMILES: Cc1cccc(/C=C(\F)C(=O)N2CCN(c3nc(OC[C@@H]4CCCN4C)nc4c3CCN(c3cccc5cccc(Cl)c35)C4)C[C@@H]2CC#N)n1]

### Example 11

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(6-methylpyridin-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(6-methylpyirin-2-yl)acrylic acid (5 mg, 0.019 mmol) were combined as solids and dissolved in DMF (0.5 mL). 1-methylimidazole (0.015 mL, 0.188 mmol) was added followed by chloro-N,N,N′,N′-tetramethylformamidinium hexafluorophospate (5 mg, 0.019 mmol) and the reaction mixture was stirred at room temperature for 1 h. The reaction mixture was directly purified by reverse phase HPLC (column: Xbridge C18, 19 mm×200 mm, 5 m particles; flow rate: 20 mL/min; column temperature: 25° C.; gradient: 68% 5:95 MeCN:H₂O with 10 mM AA/32% 95:5 MeCN:H₂O with 10 mM AA→100% 95:5 MeCN:H₂O with 10 mM AA; λ=220 nm) to afford the desired product (7.4 mg, 0.011 mmol, 56% yield) as a white solid. LC/MS (ESI) m/z: [M+H]⁺ cacld for C₃₈H₄₁ClFN₈O₂ 695.3; found 695.0; NMR: N.B. Spectrum reported as obtained; a water suppression method was used; presence of atropisomers and amide rotamers complicate analysis. ¹H NMR (500 MHz, DMSO-d₆) δ 7.92 (d, J=8.3 Hz, 1H), 7.80-7.71 (m, 2H), 7.62-7.50 (m, 3H), 7.44 (t, J=7.8 Hz, 1H), 7.38-7.30 (m, 1H), 7.24 (d, J=7.6 Hz, 1H), 6.54 (br d, J=39.4 Hz, 1H), 5.05-4.57 (m, 1H), 4.29-4.11 (m, 2H), 4.10-3.90 (m, 3H), 3.82-3.69 (m, 1H), 3.32-3.25 (m, 1H), 3.18-3.05 (m, 3H), 3.02-2.89 (m, 2H), 2.74-2.63 (m, 1H), 2.49 (br s, 3H), 2.36-2.30 (m, 3H), 2.20-2.10 (m, 1H), 1.95-1.90 (m, 1H), 1.71-1.61 (m, 2H), 1.61-1.52 (m, 1H).

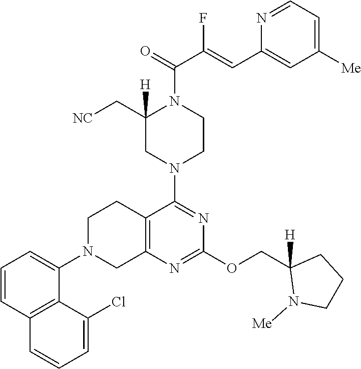 [SMILES: Cc1ccnc(/C=C(\F)C(=O)N2CCN(c3nc(OC[C@@H]4CCCN4C)nc4c3CCN(c3cccc5cccc(Cl)c35)C4)C[C@@H]2CC#N)c1]

### Example 12

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(4-methylpyridin-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(4-methylpyirin-2-yl)acrylic acid (5 mg, 0.019 mmol) were combined as solids and dissolved in DMF (0.5 mL). 1-methylimidazole (0.015 mL, 0.188 mmol) was added followed by chloro-N,N,N′,N′-tetramethylformamidinium hexafluorophospate (5 mg, 0.019 mmol) and the reaction mixture was stirred at room temperature for 1 h. The reaction mixture was directly purified by reverse phase HPLC (column: Xbridge C18, 19 mm×200 mm, 5 m particles; flow rate: 20 mL/min; column temperature: 25° C.; gradient: 69% 5:95 MeCN:H₂O with 10 mM AA/31% 95:5 MeCN:H₂O with 10 mM AA→100% 95:5 MeCN:H₂O with 10 mM AA; λ=220 nm) to afford the desired product (7.7 mg, 0.011 mmol, 59% yield) as a white solid. LC/MS (ESI) m/z: [M+H]⁺ cacld for C₃₈H₄₁ClFN₈O₂ 695.3; found 695.0; NMR: N.B. Spectrum reported as obtained; a water suppression method was used; presence of atropisomers and amide rotamers complicate analysis. ¹H NMR (500 MHz, DMSO-d₆) δ 8.49 (d, J=5.0 Hz, 1H), 7.92 (d, J=8.3 Hz, 1H), 7.79-7.70 (m, 1H), 7.62 (s, 1H), 7.60-7.50 (m, 2H), 7.47-7.42 (m, 1H), 7.38-7.30 (m, 1H), 7.22 (br d, J=5.0 Hz, 1H), 6.56 (d, J=40.6 Hz, 1H), 5.00-4.58 (m, 1H), 4.29-4.12 (m, 2H), 4.11-3.92 (m, 3H), 3.83-3.71 (m, 1H), 3.54-3.44 (m, 1H), 3.18-3.02 (m, 3H), 2.98-2.86 (m, 2H), 2.66 (s, 1H), 2.36 (s, 3H), 2.35-2.30 (m, 3H), 2.20-2.11 (m, 1H), 1.96-1.90 (m, 1H), 1.70-1.62 (m, 2H), 1.61-1.52 (m, 1H).

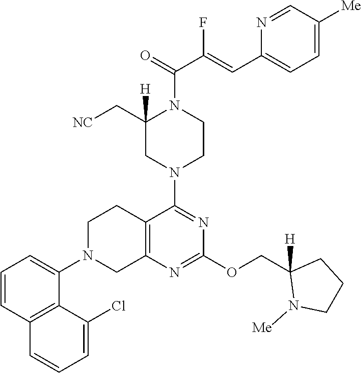 [SMILES: Cc1ccc(/C=C(\F)C(=O)N2CCN(c3nc(OC[C@@H]4CCCN4C)nc4c3CCN(c3cccc5cccc(Cl)c35)C4)C[C@@H]2CC#N)nc1]

### Example 13

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(5-methylpyridin-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(5-methylpyirin-2-yl)acrylic acid (5 mg, 0.019 mmol) were combined as solids and dissolved in DMF (0.5 mL). 1-methylimidazole (0.015 mL, 0.188 mmol) was added followed by chloro-N,N,N′,N′-tetramethylformamidinium hexafluorophospate (5 mg, 0.019 mmol) and the reaction mixture was stirred at room temperature for 1 h. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to afford the desired product (4.3 mg, 0.006 mmol, 33% yield) as a white solid. LC/MS (ESI) m/z: [M+H]⁺ cacld for C₃₈H₄₁ClFN₈O₂ 695.3; found 695.0; NMR: N.B. Spectrum reported as obtained; presence of atropisomers and amide rotamers complicate analysis. ¹H NMR (500 MHz, DMSO-d₆) δ 8.46 (s, 1H), 7.89 (br d, J=8.4 Hz, 1H), 7.74-7.66 (m, 3H), 7.57-7.49 (m, 2H), 7.45-7.41 (m, 1H), 7.36-7.29 (m, 1H), 6.56 (d, J=40.2 Hz, 1H), 5.00-4.56 (m, 1H), 4.28-4.13 (m, 2H), 4.12-3.95 (m, 3H), 3.63-3.55 (m, 2H), 3.52-3.43 (m, 1H), 3.34-3.26 (m, 1H), 3.14-3.03 (m, 3H), 3.01-2.86 (m, 2H), 2.72-2.58 (m, 1H), 2.40-2.34 (m, 3H), 2.31 (s, 3H), 2.29-2.21 (m, 1H), 2.00-1.90 (m, 1H), 1.72-1.62 (m, 2H), 1.62-1.51 (m, 1H).

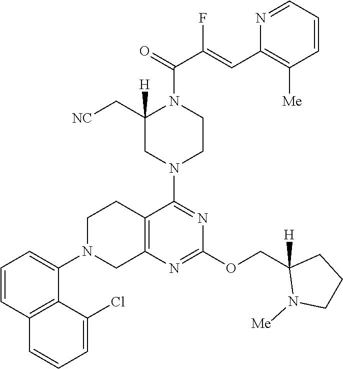 [SMILES: Cc1cccnc1/C=C(\F)C(=O)N1CCN(c2nc(OC[C@@H]3CCCN3C)nc3c2CCN(c2cccc4cccc(Cl)c24)C3)C[C@@H]1CC#N]

### Example 14

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(3-methylpyridin-2-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(3-methylpyirin-2-yl)acrylic acid (5 mg, 0.019 mmol) were combined as solids and dissolved in DMF (0.5 mL). 1-methylimidazole (0.015 mL, 0.188 mmol) was added followed by chloro-N,N,N′,N′-tetramethylformamidinium hexafluorophospate (5 mg, 0.019 mmol) and the reaction mixture was stirred at room temperature for 1 h. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to afford the desired product (4.4 mg, 0.006 mmol, 34% yield) as a white solid. LC/MS (ESI) m/z: [M+H]⁺ cacld for C₃₈H₄₁ClFN₈O₂ 695.3; found 695.0; NMR: N.B. Spectrum reported as obtained; presence of atropisomers and amide rotamers complicate analysis. ¹H NMR (500 MHz, DMSO-d₆) δ 8.44 (br d, J=4.5 Hz, 1H), 7.89 (d, J=8.3 Hz, 1H), 7.74-7.70 (m, 1H), 7.67 (d, J=7.5 Hz, 1H), 7.58-7.49 (m, 2H), 7.46-7.40 (m, 1H), 7.36-7.30 (m, 1H), 7.27 (dd, J=7.5, 4.5 Hz, 1H), 6.70 (d, J=36.3 Hz, 1H), 4.98-4.60 (m, 1H), 4.29-4.21 (m, 1H), 4.20-4.03 (m, 3H), 4.01-3.90 (m, 1H), 3.82-3.74 (m, 1H), 3.58-3.46 (m, 1H), 3.37-3.30 (m, 1H), 3.14-3.04 (m, 3H), 3.03-2.88 (m, 2H), 2.74-2.63 (m, 1H), 2.41-2.34 (m, 3H), 2.31 (s, 3H), 2.30-2.25 (m, 1H), 1.99-1.90 (m, 1H), 1.74-1.63 (m, 2H), 1.62-1.51 (m, 1H).

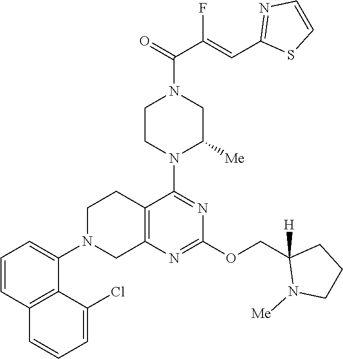 [SMILES: C[C@H]1CN(C(=O)/C(F)=C/c2nccs2)CCN1c1nc(OC[C@@H]2CCCN2C)nc2c1CCN(c1cccc3cccc(Cl)c13)C2]

### Example 15

### (Z)-1-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-3-methylpiperazin-1-yl)-2-fluoro-3-(thiazol-2-yl)prop-2-en-1-one

7-(8-chloronaphthalen-1-yl)-4-((S)-2-methylpiperazin-1-yl)-2-(((S)-1-methylpyrrolidin-2-yl)methoxy)-5,6,7,8-tetrahydropyrido[3,4-d]pyrimidine (3 mg, 0.006 mmol) and (Z)-2-fluoro-3-(thiazol-2-yl)acrylic acid (3 mg, 0.018 mmol) were combined as solids and dissolved in DMF (0.3 mL). 1-methylimidazole (0.003 mL, 0.035 mmol) was added followed by chloro-N,N,N′,N′-tetramethylformamidinium hexafluorophospate (5 mg, 0.018 mmol) and the reaction mixture was stirred at room temperature for 5 min. The reaction mixture was directly purified by reverse phase HPLC (column: Xbridge C18, 19 mm×200 mm, 5 m particles; flow rate: 20 mL/min; column temperature: 25° C.; gradient: 69% 5:95 MeCN:H₂O with 10 mM AA/31% 95:5 MeCN:H₂O with 10 mM AA→100% 95:5 MeCN:H₂O with 10 mM AA; λ=220 nm) to afford the desired product (0.6 mg, 0.9 μmol, 15% yield) as a white solid. LC/MS (ESI) m/z: [M+H]⁺ cacld for C₃₄H₃₈ClFN₇O₂S 662.2; found 662.2.

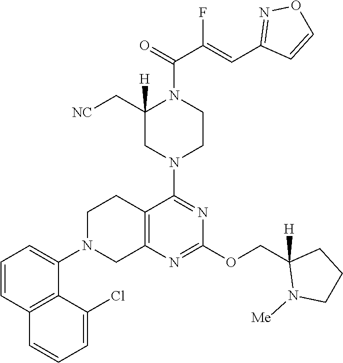 [SMILES: CN1CCC[C@H]1COc1nc2c(c(N3CCN(C(=O)/C(F)=C/c4ccon4)[C@@H](CC#N)C3)n1)CCN(c1cccc3cccc(Cl)c13)C2]

### Example 16

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(isoxazol-3-yl)acryloyl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(isoxazol-3-yl)acrylic acid, Na+ (7 mg, 0.038 mmol) were combined as solids and dissolved in DMF (1.0 mL). DIPEA (10 μL, 0.056 mmol) was added followed by COMU® ((1-Cyano-2-ethoxy-2-oxoethylidenaminooxy)dimethylamino-morpholino-carbenium hexafluorophosphate) (12 mg, 0.028 mmol). The reaction mixture was stirred at room temperature overnight. The solution directly purified by HPLC (column: Xbridge C18, 19 mm×200 mm, 5 m particles; flow rate: 20 mL/min; column temperature: 25° C.; gradient: 87% 5:95 MeCN:H₂O with 0.1% TFA/13% 95:5 MeCN:H₂O with 0.1% TFA→100% 95:5 MeCN:H₂O with 0.1% TFA; λ=220 nm) to afford the desired product as a bis-TFA salt (8.7 mg, 0.010 mmol, 25% yield). LC/MS (ESI) m/z: [M+H]⁺ cacld for C₃₅H₃₇ClFN₈O₃ 671.3; found 671.2; NMR: Spectrum reported as obtained; a water suppression method was used; presence of atropisomers and amide rotamers complicate analysis. ¹H NMR (500 MHz, DMSO-d₆) δ 9.13-8.91 (m, 1H), 7.93 (d, J=8.3 Hz, 1H), 7.84-7.67 (m, 1H), 7.65-7.49 (m, 2H), 7.49-7.42 (m, 1H), 7.42-7.32 (m, 1H), 6.91-6.87 (m, 1H), 6.77 (d, J=37.4 Hz, 1H), 5.07-4.82 (m, 1H), 4.65-4.51 (m, 1H), 4.47-4.38 (m, 1H), 4.24-4.15 (m, 2H), 4.11-3.99 (m, 2H), 3.88-3.71 (m, 3H), 3.70-3.45 (m, 2H), 3.41-3.27 (m, 1H), 3.26-3.09 (m, 1H), 2.98-2.87 (m, 1H), 2.81-2.69 (m, 1H), 2.62-2.52 (m, 1H), 2.30-2.17 (m, 1H), 2.11-2.01 (m, 1H), 1.94-1.79 (m, 2H).

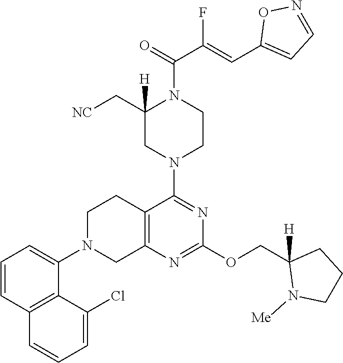 [SMILES: CN1CCC[C@H]1COc1nc2c(c(N3CCN(C(=O)/C(F)=C/c4ccno4)[C@@H](CC#N)C3)n1)CCN(c1cccc3cccc(Cl)c13)C2]

### Example 17

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(isoxazol-5-yl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (10 mg, 0.019 mmol) and (Z)-2-fluoro-3-(isoxazol-5-yl)acrylic acid, Na+(7 mg, 0.038 mmol) were combined as solids and dissolved in DMF (1.0 mL). DIPEA (10 μL, 0.056 mmol) was added followed by COMU® ((1-Cyano-2-ethoxy-2-oxoethylidenaminooxy)dimethylamino-morpholino-carbenium hexafluorophosphate) (12 mg, 0.028 mmol). The reaction mixture was stirred at room temperature overnight. The solution directly purified by HPLC (column: Xbridge C18, 19 mm×200 mm, 5 m particles; flow rate: 20 mL/min; column temperature: 25° C.; gradient: 87% 5:95 MeCN:H₂O with 0.1% TFA/13% 95:5 MeCN:H₂O with 0.1% TFA→100% 95:5 MeCN:H₂O with 0.1% TFA; λ=220 nm) to afford the desired product as a bis-TFA salt (4.4 mg, 0.005 mmol, 12% yield). LC/MS (ESI) m/z: [M+H]⁺ cacld for C₃₅H₃₇ClFN₈O₃ 671.3; found 671.2; NMR: Spectrum reported as obtained; a water suppression method was used; presence of atropisomers and amide rotamers complicate analysis. ¹H NMR (500 MHz, DMSO-d₆) δ 8.71 (s, 1H), 7.93 (d, J=8.2 Hz, 1H), 7.78-7.73 (m, 1H), 7.65-7.51 (m, 3H), 7.50-7.43 (m, 1H), 7.40-7.31 (m, 1H), 6.94 (d, J=36.6 Hz, 1H), 6.84 (s, 1H), 5.01-4.68 (m, 1H), 4.62-4.54 (m, 1H), 4.51-4.41 (m, 1H), 4.28-4.13 (m, 1H), 4.12-3.98 (m, 1H), 3.84-3.71 (m, 1H), 3.56-3.45 (m, 1H), 3.40-3.32 (m, 1H), 3.22-3.05 (m, 4H), 2.95-2.88 (m, 3H), 2.80-2.70 (m, 1H), 2.60-2.54 (m, 1H), 2.29-2.18 (m, 1H), 2.13-2.00 (m, 1H), 1.97-1.79 (m, 2H).

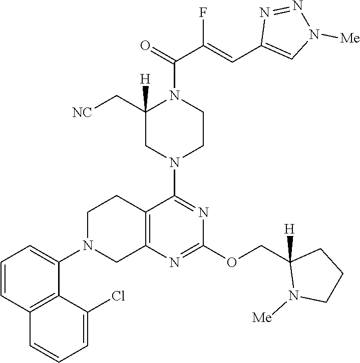 [SMILES: CN1CCC[C@H]1COc1nc2c(c(N3CCN(C(=O)/C(F)=C/c4cn(C)nn4)[C@@H](CC#N)C3)n1)CCN(c1cccc3cccc(Cl)c13)C2]

### Example 18

### 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(1-methyl-1H-1,2,3-triazol-4-yl)piperazin-2-yl)acetonitrile

2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (11 mg, 0.021 mmol) and (Z)-2-fluoro-3-(1-methyl-1H-1,2,3-triazol-4-yl)acrylic acid (10.6 mg, 0.062 mmol) were combined as solids and dissolved in DMF (1.0 mL). DIPEA (14 μL, 0.083 mmol) was added followed by COMU® ((1-Cyano-2-ethoxy-2-oxoethylidenaminooxy)dimethylamino-morpholino-carbenium hexafluorophosphate) (27 mg, 0.062 mmol). The reaction mixture was stirred at room temperature overnight. The solution directly purified by HPLC (column: Xbridge C18, 19 mm×200 mm, 5 m particles; flow rate: 20 mL/min; column temperature: 25° C.; gradient: 85% 5:95 MeCN:H₂O with 0.1% TFA/15% 95:5 MeCN:H₂O with 0.1% TFA→100% 95:5 MeCN:H₂O with 0.1% TFA; λ=220 nm) to afford the desired product as a bis-TFA salt (6.6 mg, 0.007 mmol, 12% yield). LC/MS (ESI) m/z: [M+H]⁺ cacld for C₃₅H₃₉ClFN₁₀O₂ 685.3; found 685.0; NMR: Spectrum reported as obtained; a water suppression method was used; presence of atropisomers and amide rotamers complicate analysis. ¹H NMR (500 MHz, DMSO-d₆) δ 8.42 (s, 1H), 7.93 (d, J=8.1 Hz, 1H), 7.79-7.72 (m, 1H), 7.61-7.50 (m, 2H), 7.45 (t, J=7.8 Hz, 1H), 7.39-7.31 (m, 1H), 6.78 (d, J=38.6 Hz, 1H), 4.99-4.69 (m, 1H), 4.62-4.51 (m, 1H), 4.49-4.39 (m, 1H), 4.28-4.14 (m, 1H), 4.13-4.09 (m, 3H), 4.08-3.97 (m, 1H), 3.85-3.72 (m, 1H), 3.62-3.55 (m, 1H), 3.24-3.07 (m, 4H), 2.99-2.90 (m, 3H), 2.81-2.69 (m, 1H), 2.61-2.51 (m, 1H), 2.29-2.19 (m, 1H), 2.09-2.00 (m, 1H), 1.95-1.79 (m, 2H).

### Biological Activity

### KRAS^{G12C} RAF Disruption Assay

This is a functional assay that measures activity of compounds against KRAS-G12C^{(ON)}, i.e., the active form of KRAS G12C. Recombinant GMPPNP-loaded KRAS G12C (5 nM) was treated with compound at room temperature for 20 minutes in assay buffer (50 mM Tris pH 7.5, 100 mM NaCl, 1 mM MgCl₂, 1 mM DTT, 100 ug/ml BSA). Recombinant GST-RAF1 RBD (9 nM) was added, and the reaction mixture was incubated for 20 minutes. SA-Tb (0.25 nM) was added, and the reaction mixture was incubated for 3 hours. HTRF signal was measured (PerkinElmer Envision), the signal ratio (λₑₘ 520/λₑₘ 495) was calculated, and IC₅₀ values were calculated from the dose-response curve.

The IC₅₀ values for compounds described herein are shown in Table 1.

TABLE 1

| | KRASG12C RAF | |
|---|---|---|
| | Disruption Assay | |
| Example No. | (IC₅₀) | |
| 1 | 142 | nM |
| 2 | 40261 | nM |
| 3 | 329 | nM |
| 4 | 103 | nM |
| 5 | 58 | nM |
| 6 | 89 | nM |
| 7 | 74 | nM |
| 8 | 55 | nM |
| 9 | 57000 | nM |
| 10 | 24000 | nM |
| 11 | 144 | nM |
| 12 | 129 | nM |
| 13 | 192 | nM |
| 14 | 778 | nM |
| 15 | 7500 | nM |
| 16 | 63700 | nM |
| 17 | 10500 | nM |
| 18 | 92600 | nM |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

All of the references cited herein are incorporated herein by reference in their entireties.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A compound of formula (I): 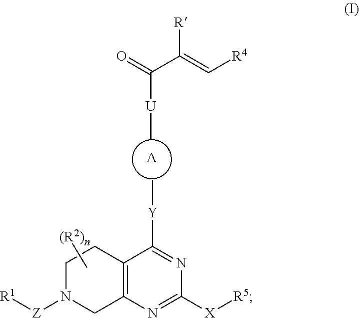
or a pharmaceutically acceptable salt thereof, wherein:
U is a bond or NH;
n is 0, 1, or 2;
Z is a bond, C(O), or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl;
each R² is independently selected from C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, hydroxy, and oxo;
Y is a bond, O, NR^{g}(CR^{e}R^{f})ₘ, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, halo, haloC₁-C₃alkyl, amino, aminoC₁-C₃alkyl, hydroxy, hydroxyC₁-C₃alkyl, and oxo; R′ is halo;
R⁴ is a five- or six-membered aromatic ring optionally containing one, two, or three heteroatoms independently selected from nitrogen, oxygen, and sulfur; wherein the ring is optionally substituted with one, two, or three substituents independently selected from C₂-C₄alkenyl, C₁-C₃alkyl, cyano, cyanoC₁-C₃alkyl, halo, haloC₁-C₃alkoxy, haloC₁-C₃alkyl, nitro, and oxo;
X is O or NR¹⁶, wherein R¹⁶ is hydrogen or C₁-C₃alkyl;
R⁵ is selected from hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from C₁-C₃alkoxy, C₁-C₃alkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, aminoC₁-C₃alkyl, hydroxy, and hydroxyC₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

2. The compound of claim 1, or a pharmaceutically acceptable salt thereof, wherein Y is a bond.

3. The compound of claim 1 or 2, or a pharmaceutically acceptable salt thereof, wherein A is a four- to nine-membered monocyclic or bicyclic bridged or fused saturated ring system optionally containing one or two nitrogen atoms.

4. The compound of any one of claims 1 to 3, or a pharmaceutically acceptable salt thereof, wherein A-U is 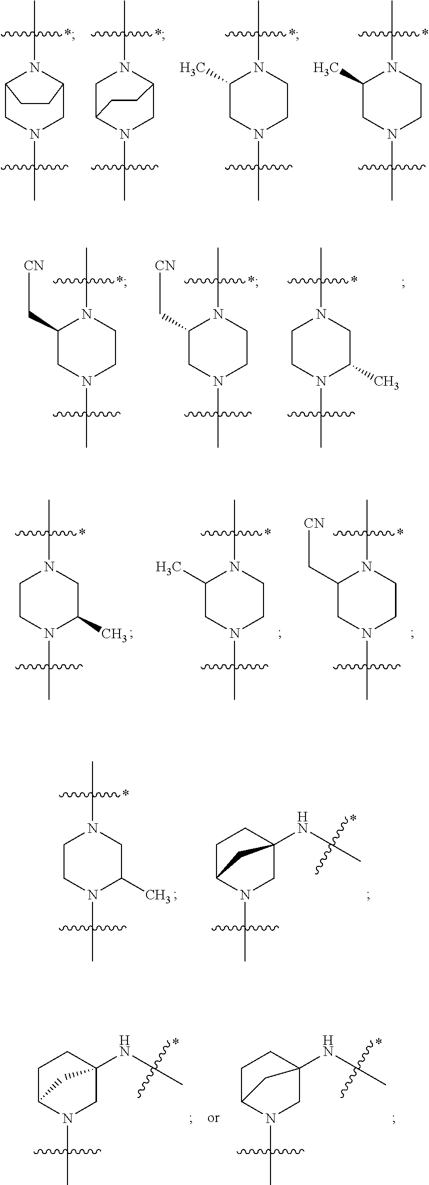
wherein [symbol]
represents the point of attachment to the carbonyl group; and [symbol]
represents the point of attachment to Y.

5. The compounds of any one of claims 1 to 4, or a pharmaceutically acceptable salt thereof,
wherein A-U is 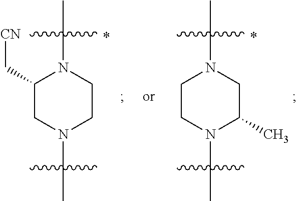
wherein [symbol]
represents the point of attachment to the carbonyl group; and [symbol]
represents the point of attachment to Y.

6. The compound of any one of claims 1 to 5, or a pharmaceutically acceptable salt thereof, wherein n is 0.

7. The compound of any one of claims 1 to 6, or a pharmaceutically acceptable salt thereof, wherein R⁴ is selected from imidazolyl, isothiazolyl, isoxazolyl, oxazolyl, phenyl, pyridazinyl, pyridinyl, pyrimidinyl, pyrazolyl, thiazolyl, and triazolyl, wherein each ring is optionally substituted with one, two, or three groups independently selected from C₂-C₄alkenyl, C₁-C₃alkyl, halo, haloC₁-C₃alkoxy, haloC₁-C₃alkyl, nitro, and oxo.

8. The compound of any one of claims 1 to 7, or a pharmaceutically acceptable salt thereof, wherein R⁴ is selected from imidazolyl, isothiazolyl, isoxazolyl, oxazolyl, pyridinyl, pyrimidinyl, thiazolyl, and triazolyl, wherein each ring is optionally substituted with a methyl or halo.

9. The compound of any one of claims 1 to 8, or a pharmaceutically acceptable salt thereof, wherein X is O.

10. The compound of any one of claims 1 to 9, or a pharmaceutically acceptable salt thereof, wherein R⁵ is selected from: 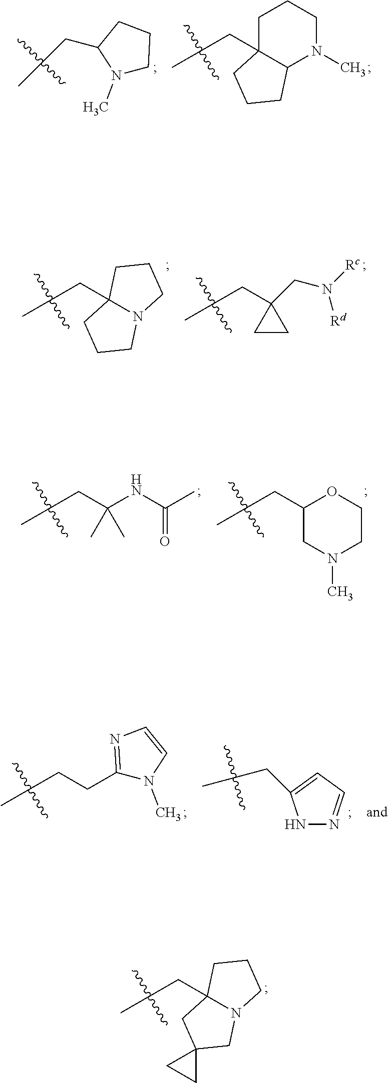
wherein each ring is optionally substituted with 1, 2, or 3 groups independently selected from C₁-C₃alkoxy, C₁-C₃alkoxyC₁-C₃alkyl, C₁-C₃alkyl, benzyl, halo, haloC₁-C₃alkyl, hydroxy, hydroxyC₁-C₃alkyl, and oxo.

11. The compound of any one of claims 1 to 10, or a pharmaceutically acceptable salt thereof, wherein R⁵ is —(C₁-C₃alkyl)-R⁶, wherein R⁶ is a three- to five-membered monocyclic ring system, an eight- or nine-membered bicyclic fused saturated ring system, or a ten-membered tricyclic saturated ring system, wherein each ring system optionally contains one nitrogen atom, and wherein each ring system is optionally substituted with one or two groups independently selected from C₁-C₃alkyl, halo, and (4- to 6-membered heterocyclyl)C₁-C₃alkyl; wherein the heterocyclyl part of the (4- to 6-membered heterocyclyl)C₁-C₃alkyl is further optionally substituted with a halo group.

12. The compound of any one of claims 1 to 11, or a pharmaceutically acceptable salt thereof,
wherein R⁵ is 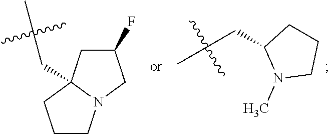
and [symbol] represents the point of attachment to X.

13. The compound of any one of claims 1 to 12, or a pharmaceutically acceptable salt thereof, wherein R⁵ is 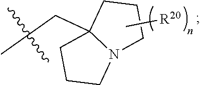
wherein n is 0, 1, or 2;
each R²⁰ is halo; and [symbol]
represents the point of attachment to X.

14. The compound of any one of claims 1 to 13, or a pharmaceutically acceptable salt thereof, wherein Z is a bond.

15. The compound of any one of claims 1 to 14, wherein R¹ is a monocyclic heteroaryl ring containing one, two, or three nitrogen atoms, wherein the ring is optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

16. The compound of any one of claims 1 to 15, wherein R¹ is 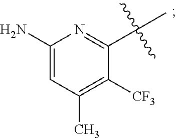
wherein [symbol] denotes the point of attachment to the parent molecular moiety.

17. The compound of any one of claims 1 to 14, or a pharmaceutically acceptable salt thereof, wherein R¹ is C₆-C₁₀aryl optionally substituted with one, two, three, four, or five substituents independently selected from C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, hydroxy, and hydroxyC₁-C₃alkyl.

18. The compound of any one of claims 1 to 14 and 17, or a pharmaceutically acceptable salt thereof, wherein R¹ is naphtyl substituted with one, two, three, four, or five substituents independently selected from C₁-C₃alkyl, C₂-C₄alkynyl, halo, and hydroxy.

19. The compound of any one of claims 1 to 14, 17, and 18, or a pharmaceutically acceptable salt thereof, wherein R¹ is naphthyl, wherein the naphthyl is substituted with one, two, or three groups independently selected from C₂-C₄alkynyl, halo, and hydroxy.

20. The compound of any one of claims 1 to 14, and 17 to 19, or a pharmaceutically acceptable salt thereof, wherein R¹ is 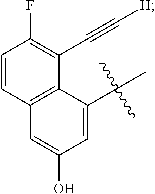
wherein [symbol] denotes the point of attachment to the parent molecular moiety.

21. The compound of any one of claims 1 to 20, or a pharmaceutically acceptable salt thereof, wherein R′ is fluoro.

22. The compound of any one of claims 1 to 20, or a pharmaceutically acceptable salt thereof, wherein R′ is chloro.

23. A compound selected from 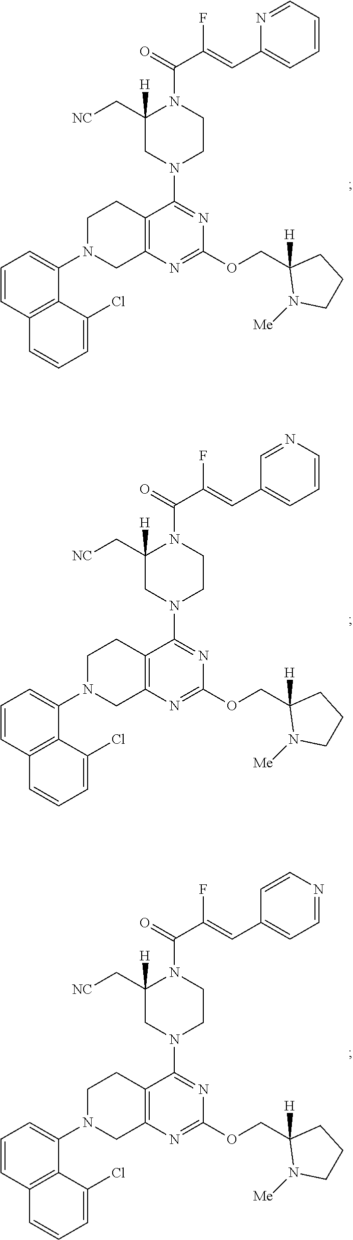 [SMILES: CN1CCC[C@H]1COc1nc2c(c(N3CCN(C(=O)/C(F)=C/c4ccccn4)[C@@H](CC#N)C3)n1)CCN(c1cccc3cccc(Cl)c13)C2 ; CN1CCC[C@H]1COc1nc2c(c(N3CCN(C(=O)/C(F)=C/c4cccnc4)[C@@H](CC#N)C3)n1)CCN(c1cccc3cccc(Cl)c13)C2 ; CN1CCC[C@H]1COc1nc2c(c(N3CCN(C(=O)/C(F)=C/c4ccncc4)[C@@H](CC#N)C3)n1)CCN(c1cccc3cccc(Cl)c13)C2] 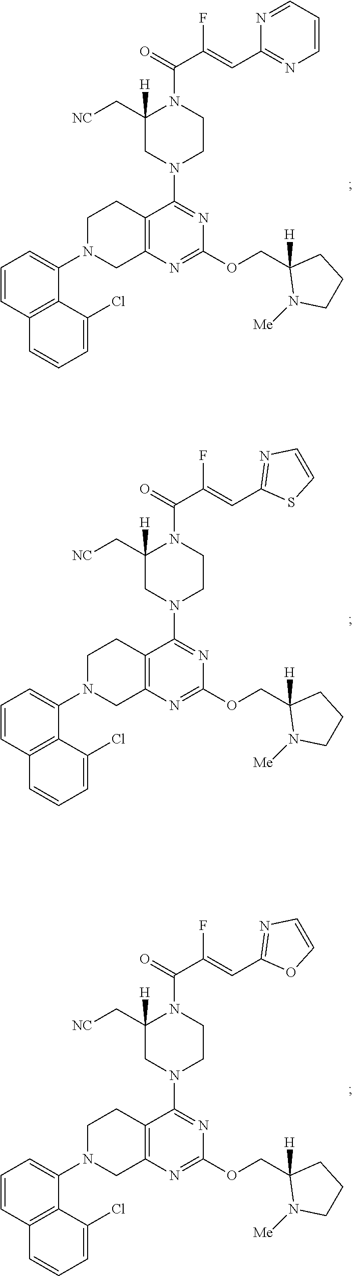 [SMILES: CN1CCC[C@H]1COc1nc2c(c(N3CCN(C(=O)/C(F)=C/c4ncccn4)[C@@H](CC#N)C3)n1)CCN(c1cccc3cccc(Cl)c13)C2 ; CN1CCC[C@H]1COc1nc2c(c(N3CCN(C(=O)/C(F)=C/c4nccs4)[C@@H](CC#N)C3)n1)CCN(c1cccc3cccc(Cl)c13)C2 ; CN1CCC[C@H]1COc1nc2c(c(N3CCN(C(=O)/C(F)=C/c4ncco4)[C@@H](CC#N)C3)n1)CCN(c1cccc3cccc(Cl)c13)C2] 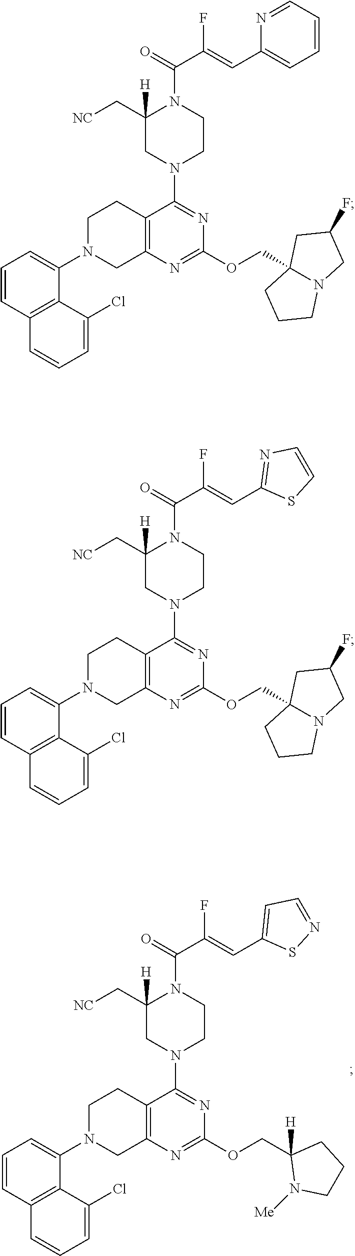 [SMILES: N#CC[C@H]1CN(c2nc(OC[C@@]34CCCN3C[C@H](F)C4)nc3c2CCN(c2cccc4cccc(Cl)c24)C3)CCN1C(=O)/C(F)=C/c1ccccn1 ; N#CC[C@H]1CN(c2nc(OC[C@@]34CCCN3C[C@H](F)C4)nc3c2CCN(c2cccc4cccc(Cl)c24)C3)CCN1C(=O)/C(F)=C/c1nccs1 ; CN1CCC[C@H]1COc1nc2c(c(N3CCN(C(=O)/C(F)=C/c4ccns4)[C@@H](CC#N)C3)n1)CCN(c1cccc3cccc(Cl)c13)C2] 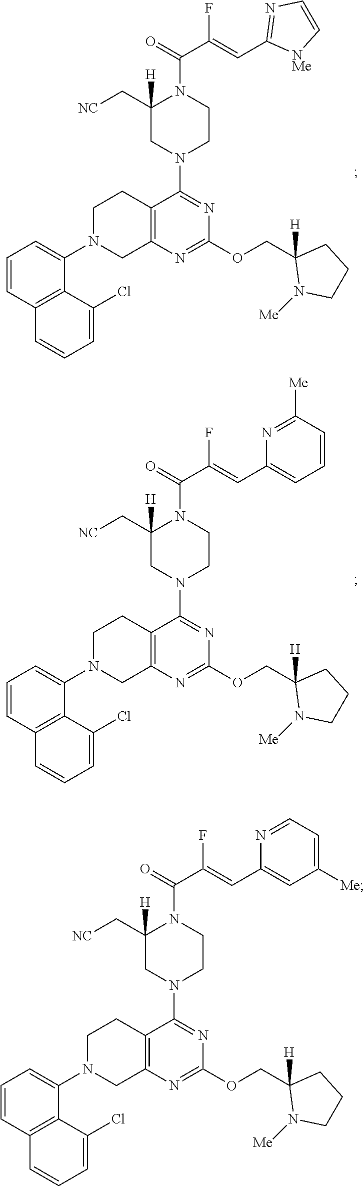 [SMILES: CN1CCC[C@H]1COc1nc2c(c(N3CCN(C(=O)/C(F)=C/c4nccn4C)[C@@H](CC#N)C3)n1)CCN(c1cccc3cccc(Cl)c13)C2 ; Cc1cccc(/C=C(\F)C(=O)N2CCN(c3nc(OC[C@@H]4CCCN4C)nc4c3CCN(c3cccc5cccc(Cl)c35)C4)C[C@@H]2CC#N)n1 ; Cc1ccnc(/C=C(\F)C(=O)N2CCN(c3nc(OC[C@@H]4CCCN4C)nc4c3CCN(c3cccc5cccc(Cl)c35)C4)C[C@@H]2CC#N)c1] 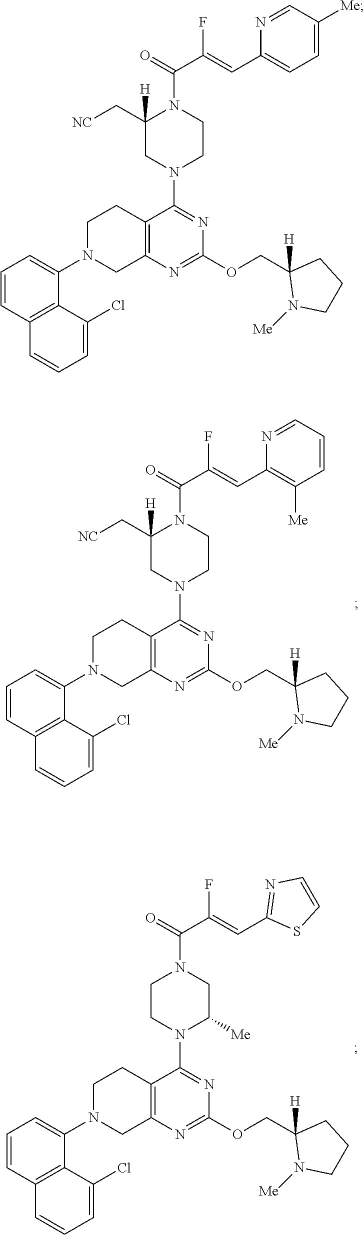 [SMILES: Cc1ccc(/C=C(\F)C(=O)N2CCN(c3nc(OC[C@@H]4CCCN4C)nc4c3CCN(c3cccc5cccc(Cl)c35)C4)C[C@@H]2CC#N)nc1 ; Cc1cccnc1/C=C(\F)C(=O)N1CCN(c2nc(OC[C@@H]3CCCN3C)nc3c2CCN(c2cccc4cccc(Cl)c24)C3)C[C@@H]1CC#N ; C[C@H]1CN(C(=O)/C(F)=C/c2nccs2)CCN1c1nc(OC[C@@H]2CCCN2C)nc2c1CCN(c1cccc3cccc(Cl)c13)C2] 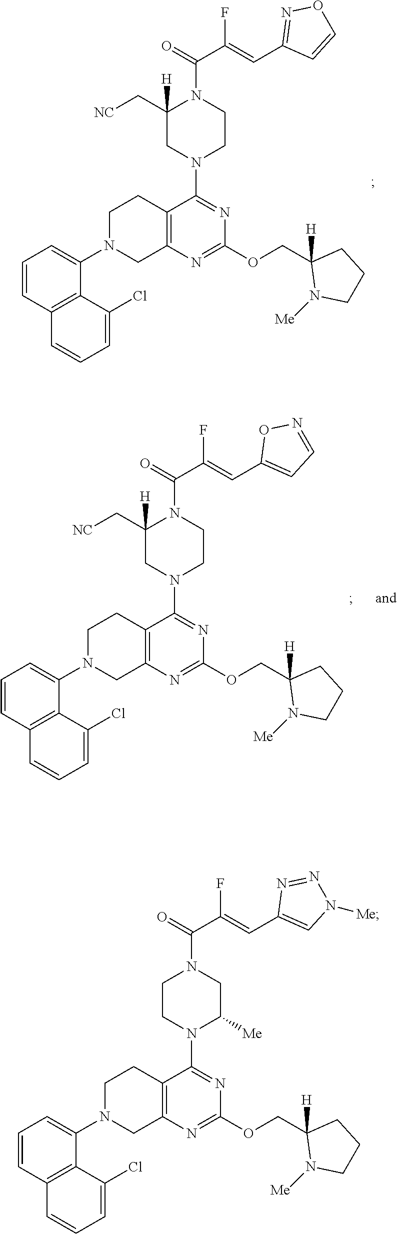 [SMILES: CN1CCC[C@H]1COc1nc2c(c(N3CCN(C(=O)/C(F)=C/c4ccon4)[C@@H](CC#N)C3)n1)CCN(c1cccc3cccc(Cl)c13)C2 ; CN1CCC[C@H]1COc1nc2c(c(N3CCN(C(=O)/C(F)=C/c4ccno4)[C@@H](CC#N)C3)n1)CCN(c1cccc3cccc(Cl)c13)C2 ; C[C@H]1CN(C(=O)/C(F)=C/c2cn(C)nn2)CCN1c1nc(OC[C@@H]2CCCN2C)nc2c1CCN(c1cccc3cccc(Cl)c13)C2]
or a pharmaceutically acceptable salt thereof.

24. A compound selected from:
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-3-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyridin-4-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(pyrimidin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(oxazol-2-yl)acryloyl)piperazin-2-yl)acetonitrile;
2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(pyridin-2-yl)acryloyl)piperazin-2-yl)acetonitrile;
2-((S)-4-(7-(8-chloronaphthalen-1-yl)-2 (((2R,7aS)-2-fluorotetrahydro-1H-pyrrolizin-7a(5H)-yl)methoxy)-5,6,7,8-tetrahydropyrido[3,4-d]pyrimidin-4-yl)-1-((Z)-2-fluoro-3-(thiazol-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoro-3-(isothiazol-5-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(1-methyl-1H-imidazol-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(6-methylpyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(4-methylpyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(5-methylpyridin-2-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(3-methylpyridin-2-yl)acryloyl)piperazin-2-yl)acetonitrile;
(Z)-1-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-3-methylpiperazin-1-yl)-2-fluoro-3-(thiazol-2-yl)prop-2-en-1-one;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(isoxazol-3-yl)acryloyl)piperazin-2-yl)acetonitrile;
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(isoxazol-5-yl)piperazin-2-yl)acetonitrile; and
2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((Z)-2-fluoro-3-(1-methyl-1H-1,2,3-triazol-4-yl)piperazin-2-yl)acetonitrile
or a pharmaceutically acceptable salt thereof.

25. A pharmaceutical composition comprising a compound of any one of claims 1 to 24, or a pharmaceutically acceptable salt thereof, and one or more pharmaceutically acceptable excipients.

26. An oral dosage form comprising a compound of any one of claims 1 to 24, or a pharmaceutically acceptable salt thereof, and one or more pharmaceutically acceptable excipients.

27. A method of treating cancer expressing KRAS G12C, G12D and/or G12V mutation in a subject in need thereof, the method comprising administering to the subject a compound of any one of claims 1 to 24, or a pharmaceutically acceptable salt thereof.

28. A method of treating cancer expressing KRAS G12C mutation in a subject in need thereof, the method comprising administering to the subject a compound of any one of claims 1 to 24, or a pharmaceutically acceptable salt thereof.

29. A method for treating a cancer susceptible to KRAS G12C inhibition in a subject in need thereof, the method comprising administering to the subject a compound of any one of claims 1 to 24, or a pharmaceutically acceptable salt thereof.

30. A method for treating a cancer in a subject in need thereof, the method comprising administering to the subject a compound of any one of claims 1 to 24, or a pharmaceutically acceptable salt thereof, wherein the cancer is lung cancer, colorectal cancer, pancreatic cancer, breast cancer, bladder cancer, cervical cancer, ovarian cancer, gastric cancer or cancer of the uterus.

31. A method for treating a cancer in a subject in need thereof, the method comprising administering to the subject a compound of any one of claims 1 to 24, or a pharmaceutically acceptable salt thereof, wherein the cancer is non-small cell lung cancer.
